SEQUENCE LISTING (8 sequences)

SEQ ID NO: 1 - DNA, 1226 bp, Homo sapiens
  CDS (57)..(965)
ctgggccgcgaggcgcggagcttgggagcggagcccaggccgtgccgcgcggcgccatgaagggcaaggaggagaaggagggcggcgcacggctgggcgctggcggcggaagccccgagaagagcccgagcgcgcaggagctcaaggagcagggcaatcgtctgttcgtgggccgaaagtacccggaggcggcggcctgctacggccgcgcgatcacccggaacccgctggtggccgtgtattacaccaaccgggccttgtgctacctgaagatgcagcagcacgagcaggccctggccgactgccggcgcgccctggagctggacgggcagtctgtgaaggcgcacttcttcctggggcagtgccagctggagatggagagctatgatgaggccatcgccaatctgcagcgagcttacagcctggccaaggagcagcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgacccggtgaccgggagccccctgacccaggaacagttcatccccaacttggctatgaaggaggttattgacgcattcatctctgagaatggctgggtggaggactactgaggttccctgccctacctggcgtcctggtccaggggagccctgggcagaagcccccggcccctaaacatagtttatgtttttggccaccccgaccgcttcccccaagttctgctgttggactctggactgtttcccctctcagcatcgcttttgctgggccgtgattgtcccctttgtgggctggaaaagcaggtgagggtgggctgggctgaggccattgccgccactatctgtgtaataaaatccgtgagcacgaaa

SEQ ID NO: 2 - protein, 303 aa, Homo sapiens
MKGKEEKEGGARLGAGGGSPEKSPSAQELKEQGNRLFVGRKYPEAAACYGRAITRNPLVAVYYTNRALCYLKMQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTGSPLTQEQFIPNLAMKEVIDAFISENGWVEDY

SEQ ID NO: 3 - DNA, 2276 bp, Homo sapiens
  CDS (79)..(2016)
ctcattgaactcgcctgcagctcttgggttttttgtggcttccttcgttattggagccaggcctacaccccagcaaccatgtccaagggacctgcagttggtattgatcttggcaccacctactcttgtgtgggtgttttccagcacggaaaagtcgagataattgccaatgatcagggaaaccgaaccactccaagctatgtcgcctttacggacactgaacggttgatcggtgatgccgcaaagaatcaagttgcaatgaaccccaccaacacagtttttgatgccaaacgtctgattggacgcagatttgatgatgctgttgtccagtctgatatgaaacattggccctttatggtggtgaatgatgctggcaggcccaaggtccaagtagaatacaagggagagaccaaaagcttctatccagaggaggtgtcttctatggttctgacaaagatgaaggaaattgcagaagcctaccttgggaagactgttaccaatgctgtggtcacagtgccagcttactttaatgactctcagcgtcaggctaccaaagatgctggaactattgctggtctcaatgtacttagaattattaatgagccaactgctgctgctattgcttacggcttagacaaaaaggttggagcagaaagaaacgtgctcatctttgacctgggaggtggcacttttgatgtgtcaatcctcactattgaggatggaatctttgaggtcaagtctacagctggagacacccacttgggtggagaagattttgacaaccgaatggtcaaccattttattgctgagtttaagcgcaagcataagaaggacatcagtgagaacaagagagctgtaagacgcctccgtactgcttgtgaacgtgctaagcgtaccctctcttccagcacccaggccagtattgagatcgattctctctatgaaggaatcgacttctatacctccattacccgtgcccgatttgaagaactgaatgctgacctgttccgtggcaccctggacccagtagagaaagcccttcgagatgccaaactagacaagtcacagattcatgatattgtcctggttggtggttctactcgtatccccaagattcagaagcttctccaagacttcttcaatggaaaagaactgaataagagcatcaaccctgatgaagctgttgcttatggtgcagctgtccaggcagccatcttgtctggagacaagtctgagaatgttcaagatttgctgctcttggatgtcactcctctttcccttggtattgaaactgctggtggagtcatgactgtcctcatcaagcgtaataccaccattcctaccaagcagacacagaccttcactacctattctgacaaccagcctggtgtgcttattcaggtttatgaaggcgagcgtgccatgacaaaggataacaacctgcttggcaagtttgaactcacaggcatacctcctgcaccccgaggtgttcctcagattgaagtcacttttgacattgatgccaatggtatactcaatgtctctgctgtggacaagagtacgggaaaagagaacaagattactatcactaatgacaagggccgtttgagcaaggaagacattgaacgtatggtccaggaagctgagaagtacaaagctgaagatgagaagcagagggacaaggtgtcatccaagaattcacttgagtcctatgccttcaacatgaaagcaactgttgaagatgagaaacttcaaggcaagattaacgatgaggacaaacagaagattctggacaagtgtaatgaaattatcaactggcttgataagaatcagactgctgagaaggaagaatttgaacatcaacagaaagagctggagaaagtttgcaaccccatcatcaccaagctgtaccagagtgcaggaggcatgccaggaggaatgcctgggggatttcctggtggtggagctcctccctctggtggtgcttcctcagggcccaccattgaagaggttgattaagccaaccaagtgtagatgtagcattgttccacacatttaaaacatttgaaggacctaaattcgtagcaaattctgtggcagttttaaaaagttaagctgctatagtaagttactgggcattctcaatacttgaatatggaacatatgcacaggggaaggaaataacattgcactttataaacactgtattgtaagtggaaaatgcaatgtcttaaataaaactatttaaaattggcaccataaaaaaaaaaaaaaaa

SEQ ID NO: 4 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 5 - DNA, 2379 bp, Homo sapiens
  CDS (178)..(2100)
cctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccggcatggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgatcggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggcggacaagaagaaggttctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtggattaggggcctttccaagattgctgtttttgttttggagcttcaagactttgcatttcctagtatttctgtttgtcagttctcaatttcctgtgtttgcaatgttgaaattttttggtgaagtactgaacttgctttttttccggtttctacatgcagagatgaatttatactgccatcttacgactatttcttctttttaatacacttaactcaggccattttttaagttggttacttcaaagtaaataaactttaaaattcaaaaaaaaaaaaaaaaaa

SEQ ID NO: 6 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 7 - DNA, 2577 bp, Homo sapiens
  CDS (98)..(2269)
ctctctcgagtcactccggcgcagtgttgggactgtctgggtatcggaaagcaagcctacgttgctcactattacgtataatccttttcttttcaagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcaggctggtgcagacatctccatgattgggcagtttggtgttggcttttattctgcctacttggtggcagagaaagtggttgtgatcacaaagcacaacgatgatgaacagtatgcttgggagtcttctgctggaggttccttcactgtgcgtgctgaccatggtgagcccattggcaggggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaagcccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgtttttatacaaaaaaaaaaaaaaaaaaaaa

SEQ ID NO: 8 - protein, 724 aa, Homo sapiens
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

(19) United States
(12) Patent Application Publication
(10) Pub. No.: US 2005/0186587 A1
(43) Pub. Date: Aug. 25, 2005

(54) Method for screening a substance which can suppress degradation of a nuclear receptor or a substance which can promote degradation of a nuclear receptor

(75) Inventors: Junn Yanagisawa, Ibaraki (JP); Yukiyo Tateishi, Ibaraki (JP)

Correspondence Address: LAHIVE & COCKFIELD, LLP., 28 STATE STREET, BOSTON, MA, 02109, US

(73) Assignee: Interite, Inc., Shibuya-ku (JP)

(21) Appl. No.: 10/926,802

(22) Filed: Aug. 25, 2004

(30) Foreign Application Priority Data
Feb. 23, 2004 (JP) 2004-047126

(51) Int. Cl. C12Q 1/68; G01N 33/53; G01N 33/567; G06F 19/00; G01N 33/48; G01N 33/50

(52) U.S. Cl. 435/6; 435/7.2; 702/19

(57) ABSTRACT

A method is provided for screening a substance which can suppress or promote degradation of a nuclear receptor, comprising the steps of: judging whether or not a test substance can suppress or promote binding of a CHIP with a nuclear receptor via a chaperone protein; and screening a test substance which can suppress the binding as a substance which can suppress degradation of a nuclear receptor or screening a test substance which can promote the binding as a substance which can promote degradation of a nuclear receptor.

### RELATED APPLICATIONS

This application claims priority to Japanese Application No. 2004-047126 filed Feb. 23, 2004. This application is hereby incorporated herein by reference in its entirety.

### BACKGROUND OF THE INVENTION

1. Field of the Invention

The present invention relates to a method for screening a substance which can suppress degradation of a nuclear receptor or a substance which can promote degradation of a nuclear receptor.

2. Description of the Related Art

Because nuclear receptors are closely involved in such diseases as diabetes, osteoporosis, hypertension, hormone-dependent cancers (such as breast cancer, uterine cancer and prostate cancer) and the like, agonists and antagonists to nuclear receptors are under scrutiny as therapeutic drugs.

However, the functions of nuclear receptors differ depending on the tissue, and their responsiveness to ligands also differs depending on the tissue. Consequently, agonists and antagonists to nuclear receptors may have therapeutic effects in certain tissues but side-effects in other tissues. For example, in the case of estrogen receptors, tamoxifen suppresses proliferation of estrogen-dependent breast cancers and has the advantage of not causing bone loss because it does not act as an antagonist in bone, but it has the side-effect of promoting proliferation of estrogen-dependent uterine cancers. Thus, there is a need for different approaches to suppressing the functions of nuclear receptors, and one that has been considered is controlling the functions of nuclear receptors by suppressing or promoting degradation of nuclear receptors.

It has been reported that Hsp90 binds to estrogen receptor alpha (ERα) (Beliakoff, J. et al., Clinical Cancer Research, 2003, 9(13), p. 4961-71; Lee, M O. et al., Molecular and Cellular Endocrinology, 2002, 188(1-2), p. 47-54), and that Hsp70 binds to estrogen receptor alpha (ERα) (Sai Padma, A. et al., Journal of Cell Biochemistry, 2000, 78(4), p. 650-65 ; Klinge, C M. et al., Journal of Steroid Biochemistry and Molecular Biology, 1997, 63(4-6), p. 283-301; Landel, C C. et al., Journal of Steroid Biochemistry and Molecular Biology, 1997, 63(1-3), p. 59-73). It has also been reported that CHIP (carboxyl-terminus of Hsc70 interacting protein), which is known to bind to Hsc70, is involved in ubiquitination and degradation of CFTR (cystic-fibrosis transmembrane-conductance regulator) (Meacham, G C. et al., Nature Cell Biology, 2001, 3(1), p. 100-5), GR (glucocorticoid receptor) (Connell, P. et al., Nature Cell Biology, 2001, 3(1), p. 93-6), ErbB2 (Zhou, P. et al., Journal of Biological Chemistry, 2003, 278(16), p. 13829-37. Epub Feb. 6, 2003; Xu, W. et al., Proceedings of National Academy of Science USA., 2002, 99(20), p. 12847-52. Epub Sep. 18, 2002), androgen receptor (Cardozo, C P., Archives of Biochemistry and Biophysics, 2003, 410(1):13, p. 4-40), Smad (Li, L. et al., Molecular and Celluar Biology, 2004, 24(2), p. 856-64), Perl receptor (Imai, Y. et al., Molecular Cell, 2002, 10(1), p. 55-67) and the like.

### SUMMARY OF THE INVENTION

It is an object of the present invention to provide a method for screening a substance which can suppress degradation of a nuclear receptor and a substance which can promote degradation of a nuclear receptor.

The present invention was perfected based on the novel discovery that a CHIP binds via a chaperone protein to a nuclear receptor not bound to a nuclear receptor ligand, the nuclear receptor not bound to a nuclear receptor ligand is ubiquitinated by the CHIP bound via the chaperone protein to the nuclear receptor not bound to a nuclear receptor ligand, so that the nuclear receptor not bound to a nuclear receptor ligand is nuclear receptor ligand-independently degraded, and in order to solve the aforementioned problems, the present invention provides the following method for screening a substance which can suppress the degradation of a nuclear receptor and a substance which can promote the degradation of a nuclear receptor.
(1) A method for screening a substance which can suppress degradation of a nuclear receptor, comprising the steps of:

judging whether or not a test substance can suppress binding of a CHIP with a nuclear receptor via a chaperone protein; and

screening a test substance which can suppress the binding as a substance which can suppress degradation of a nuclear receptor.
(2) A method for screening a substance which can suppress degradation of a nuclear receptor, comprising the steps of:

judging whether or not a test substance can suppress binding of a CHIP with a chaperone protein; and

screening a test substance which can suppress the binding as a substance which can suppress degradation of a nuclear receptor.
(3) A method for screening a substance which can suppress degradation of a nuclear receptor, comprising the steps of:

judging whether or not a test substance can suppress binding of a chaperone protein with a nuclear receptor; and

screening a test substance which can suppress the binding as a substance which can suppress degradation of a nuclear receptor.
(4) A method for screening a substance which can suppress degradation of a nuclear receptor, comprising the steps of:

judging whether or not a test substance can suppress ubiquitination of a nuclear receptor by CHIP; and

screening a test substance which can suppress the ubiquitination as a substance which can suppress degradation of a nuclear receptor.
(5) A method for screening a substance which can suppress degradation of a nuclear receptor, comprising the steps of:

judging whether or not a test substance can suppress expression of a gene encoding a CHIP; and

screening a test substance which can suppress the expression as a substance which can suppress degradation of a nuclear receptor.
(6) A method for screening a substance which can suppress degradation of a nuclear receptor, comprising the steps of:

judging whether or not a test substance can suppress expression of a gene encoding a chaperone protein; and

screening a test substance which can suppress the expression as a substance which can suppress degradation of a nuclear receptor.
(7) A screening method according to any of (1) through (6), wherein the nuclear receptor is an estrogen receptor.
(8) A screening method according to any of (1) through (6), wherein the substance which can suppress degradation of a nuclear receptor is a substance which can suppress nuclear receptor ligand-independent degradation of a nuclear receptor not bound to a nuclear receptor ligand.
(9) A method for screening a substance which can promote degradation of a nuclear receptor, comprising the steps of:

judging whether or not a test substance can promote binding of a CHIP with a nuclear receptor via a chaperone protein; and

screening a test substance which can promote the binding as a substance which can promote degradation of a nuclear receptor.
(10) A method for screening a substance which can promote degradation of a nuclear receptor, comprising the steps of:

judging whether or not a test substance can promote binding of a CHIP with a chaperone protein; and

screening a test substance which can promote the binding as a substance which can promote degradation of a nuclear receptor.
(11) A method for screening a substance which can promote degradation of a nuclear receptor, comprising the steps of:

judging whether or not a test substance can promote binding of a chaperone protein with a nuclear receptor; and

screening a test substance which can promote the binding as a substance which can promote degradation of a nuclear receptor.
(12) A method for screening a substance which can promote degradation of a nuclear receptor, comprising the steps of:

judging whether or not a test substance can promote ubiquitination of a nuclear receptor by CHIP; and

screening a test substance which can promote the ubiquitination as a substance which can promote degradation of a nuclear receptor.
(13) A method for screening a substance which can promote degradation of a nuclear receptor, comprising the steps of:

judging whether or not a test substance can promote expression of a gene encoding a CHIP; and

screening a test substance which can promote the expression as a substance which can promote degradation of a nuclear receptor.
(14) A method for screening a substance which can promote degradation of a nuclear receptor, comprising the steps of:

judging whether or not a test substance can promote expression of a gene encoding a chaperone protein; and

screening a test substance which can promote the expression as a substance which can promote degradation of a nuclear receptor.
(15) A screening method according to any of (9) through (14), wherein the nuclear receptor is an estrogen receptor.
(16) A screening method according to any of claims (9) through (14), wherein the substance which can promote degradation of a nuclear receptor is a substance which can promote nuclear receptor ligand-independent degradation of a nuclear receptor not bound to a nuclear receptor ligand.

### BRIEF DESCRIPTION OF THE DRAWINGS

FIG. 1(a) shows results for detection of ubiquitinated ERα (ERα-Ub) in the presence or absence of estrogen, while FIG. 1(b) shows results for detection of estrogen receptor alpha (ERα) in the presence or absence of estrogen or the proteasome inhibitor MG132.

FIG. 2 shows results for detection of estrogen receptor alpha (ERα) or its deletion mutant ERαΔAD in the presence or absence of estrogen or the proteasome inhibitor MG132.

FIG. 3 shows results for detection of ubiquitinated ERαΔAD (Flag-ERαΔAD-Ub) in the presence or absence of estrogen or the proteasome inhibitor MG132.

FIG. 4 shows results for detection of various deletion mutants of ERα in the presence or absence of estrogen.

FIG. 5 shows results for isolation and purification from HeLa cell raw nucleus extract of a protein which binds to an estrogen receptor in the absence of estrogen.

FIG. 6 shows results for detection of ERα and CHIP in the presence or absence of ligands (estrogen, tamoxifen (OHT), fulvestrant (ICI)).

FIG. 7 shows results for detection of ERα or ERαΔAD in the presence or absence of estrogen or CHIP.

FIG. 8(a) shows results for detection of ERα or ERαΔAD in the presence and absence of OHT or CHIP, while FIG. 8(b) shows results for detection of ERα or ERαΔAD in the presence and absence of ICI or CHIP.

FIG. 9(a) shows results for detection of ERα in cells having introduced CHIP or CHIPΔUbox, while FIG. 9(b) shows transcriptional activity and transcriptional activity per amount of ERα in cells having introduced CHIP or CHIPΔUbox.

FIG. 10 shows transcriptional activity in the presence or absence of estrogen or CHIP in cells having introduced normal ERα or the thermally sensitive dominant negative mutant ERα(C447A).

FIG. 11 shows results for detection of ERα, ERα(V364E), ERα(C447A), ERαΔAD or ERα(L540Q) in the presence or absence of the proteasome inhibitor MG132.

FIG. 12 shows transcriptional activity in the presence or absence of CHIP of cells in which normal ERα and mutant ERα (mut.ERα) are co-expressed.

### DESCRIPTION OF THE PREFERRED EMBODIMENTS

The present invention is explained in detail below.

There are no particular limits on the type of test substance, which may be either a naturally occurring substance or an artificially synthesized substance. For example, the test substance may be a polymer compound, low molecular weight compound, antibody, protein, peptide, nucleic acids, sugar, inorganic salt or metal complex or a complex of these or the like, but is not limited by these. “Nucleic acids” include DNA, RNA and analogues and derivatives of these (such as peptide nucleic acids (PNA), phosphorothioate DNA and the like).

The term “nuclear receptor” mainly refers to factors which control transcription of a target gene by binding upstream from the target gene promoter in a nuclear receptor ligand-dependent fashion. However, some nuclear receptors lack nuclear receptor ligands. Consequently, even nuclear receptors which lack nuclear receptor ligands are called “nuclear receptors” if their structural and functional homology places them in the nuclear receptor gene superfamily. Nuclear receptors include estrogen receptors (ER), vitamin D receptors (VDR), peroxisome proliferator-activated receptors (PPAR), liver X receptors (LXR), retinoic acid receptors (RAR), retinoid X receptors (RXR), androgen receptors (AR), glucocorticoid receptors (GR), farnesoid x receptors (FXR), mineralcorticoid receptors (MR) and the like for example, but are not limited by these.

The nuclear receptor used in the present invention may be either a wild nuclear receptor (for example, a wild nuclear receptor derived from humans, monkeys, cows, sheep, goats, horses, pigs, rabbits, dogs, cats, mice, rats or other mammals), a mutant nuclear receptor having deletions, substitutions, additions or other mutations artificially introduced into a wild nuclear receptor, a derivative of a wild nuclear receptor or mutant nuclear receptor, or a fused protein of a wild nuclear receptor or mutant nuclear receptor with another protein or peptide or the like.

A mutant nuclear receptor is a protein comprising an amino acid sequence having one or more amino acids deleted, substituted or added in the amino acid sequence of a wild nuclear receptor, and there are no particular limits on the number of amino acids deleted, substituted or added as long as binding properties with respect to a chaperone protein are retained, with the number being one or more, and the specific range being normally 1 to 100 or preferably 1 to 50 or more preferably 1 to 25. There are also no particular limits on the locations of the amino acids deleted, substituted or added as long as binding properties with respect to a chaperone protein are retained.

There are no particular limits on the type of derivative of a wild nuclear receptor or mutant nuclear receptor as long as binding properties with respect to a chaperone protein are retained. Derivation methods include for example addition of a label substance or modification or the like with a phosphate group, methyl group, acetyl group or the like. There are no particular limits on the type of label substance, and examples include fluorescein, rhodamine, phycoerythrin, Cy pigments, Alexa pigments, BODIPY pigments and other fluorescent compounds; luminol, lucigenin, acridinium ester and other chemiluminescent compounds; alkaline phosphatase, horse radish peroxidase and other enzymes; luciferase, luciferin and other bioluminescent compounds; and ³²P, ³⁵S and other radioisotopes (RI).

There are no particular limits on the type of fused protein as long as binding properties with respect to a chaperone protein are retained. Examples of proteins or peptides to be fused include beta-galactosidase, protein A, the IgG binding region of protein A, chloramphenicol acetyltransferase, poly(Arg), poly(Glu), protein G, maltose-binding protein, glutathione S transferase (GST), polyhistidine chain (His-tag), S peptide, DNA binding protein domain, Tac antigen, thioredoxin, green fluorescent protein, hemagglutinin protein (HA-tag), FLAG-tag, Myc-tag, T7 gene 10 protein, bovine papilloma virus L1 protein, streptavidin, VSV-G-tag and the like.

The term “nuclear receptor ligand” refers to substances which control transcriptional activation of target genes of nuclear receptors by binding to the nuclear receptors. Nuclear receptor ligands may be either naturally occurring substances or artificially synthesized substances. Ligands of estrogen receptors include estrogen for example, while ligands of vitamin D receptors include vitamin D for example. Endogenous nuclear receptor ligands have not been discovered for all nuclear receptors.

A “CHIP” (Carboxy-terminus of Hsc70 interacting protein) is a molecule discovered as a co-chaperone (chaperone augmenting factor) which has at its N terminus a repeating structure called TPR (tetratricopeptide repeat) which interacts with Hsc70 and at its C terminus a structure called U-box which is characteristic of ubiquitin ligases.

The CHIP used in the present invention may be either a wild CHIP (for example, a wild CHIP derived from humans, monkeys, cows, sheep, goats, horses, pigs, rabbits, dogs, cats, mice, rats or other mammals), a mutant CHIP having deletions, substitutions, additions or other mutations artificially introduced into a wild CHIP, a derivative of a wild CHIP or mutant CHIP, or a fused protein of a wild CHIP or mutant CHIP with another protein or peptide or the like.

An example of wild CHIP is a protein comprising the amino acid sequence represented by SEQ ID NO:2. The protein comprising the amino acid sequence represented by SEQ ID NO:2 is a human-derived protein.

A mutant CHIP is a protein comprising an amino acid sequence having one or more amino acids deleted, substituted or added in the amino acid sequence of a wild CHIP, and there are no particular limits on the number of amino acids deleted, substituted or added as long as binding properties with respect to a chaperone protein are retained, with the number being one or more, and the specific range being normally 1 to 100 or preferably 1 to 50 or more preferably 1 to 25. There are also no particular limits on the locations of the amino acids deleted, substituted or added as long as binding properties of wild CHIP with respect to a chaperone protein are retained. An example of mutant CHIP is a protein comprising an amino acid sequence in which one or more amino acids are deleted, substituted or added in the amino acid sequence represented by SEQ ID NO:2, and which can bind to a chaperone protein.

There are no particular limits on the type of derivative of a wild CHIP or mutant CHIP as long as binding properties with respect to a chaperone protein are retained. Methods of derivation include for example addition of a label substance or modification with a phosphoric acid group, methyl group, acetyl group or the like. There are no particular limits on the type of label substance and specific examples are as given above.

There are no particular limits on the type of fused protein of a wild CHIP or mutant CHIP with another protein or peptide as long as binding properties with respect to a chaperone protein are retained. Specific examples of other proteins or peptides to be fused are as given above.

A “chaperone protein” has the function of maintaining the function of a protein by maintaining the normal folded structure of the protein, and examples of chaperone proteins include Hsc70, Hsp70, Hsp90 and the like, with Hsc70 being an example of a preferred chaperone protein. Hsp70 and Hsp90 are proteins which are expressed in response to a stress such as heat shock, while Hsc70 protein is a protein which is constantly present in cells.

The chaperone protein used in the present invention may be either a wild chaperone protein (for example, a wild chaperone protein derived from humans, monkeys, cows, sheep, goats, horses, pigs, rabbits, dogs, cats, mice, rats or other mammals), a mutant chaperone protein having deletions, substitutions, additions or other mutations artificially introduced into a wild chaperone protein, a derivative of a wild chaperone protein or mutant chaperone protein, or a fused protein of a wild chaperone protein or mutant chaperone protein with another protein or peptide or the like.

An example of wild Hsc70 is a protein comprising the amino acid sequence represented by SEQ ID NO:4, an example of wild Hsp70 is a protein comprising the amino acid sequence represented by SEQ ID NO:6, and an example of wild Hsp90 is a protein comprising the amino acid sequence represented by SEQ ID NO:8. The proteins comprising the amino acid sequences represented by SEQ ID NO:4, SEQ ID NO:6 and SEQ ID NO:8 are human-derived proteins.

A mutant chaperone protein is a protein comprising an amino acid sequence having one or more amino acids deleted, substituted or added in the amino acid sequence of a wild chaperone protein, and there are no particular limits on the number of amino acids deleted, substituted or added as long as binding properties with respect to a nuclear receptor and CHIP are retained, with the number being one or more, and the specific range being normally 1 to 100 or preferably 1 to 50 or more preferably 1 to 25. There are also no particular limits on the locations of the amino acids deleted, substituted or added as long as binding properties with respect to a nuclear receptor and CHIP are retained. Specific examples of mutant Hsc70, mutant Hsp70 and mutant Hsp90 are proteins comprising amino acid sequences in which one or more amino acids are deleted, substituted or added in the amino acid sequences represented by SEQ ID NO:4, SEQ ID NO:6 and SEQ ID NO:8, respectively, and which can bind to a nuclear receptor and CHIP.

There are no particular limits on the type of derivative of a wild chaperone protein or mutant chaperone protein as long as binding properties with respect to a nuclear receptor and CHIP are retained. Methods of derivation include for example addition of a label substance or modification with a phosphoric acid group, methyl group, acetyl group or the like. There are no particular limits on the type of label substance and specific examples are as given above.

There are no particular limits on the type of fused protein of a wild chaperone protein or mutant chaperone protein with another protein or peptide as long as binding properties with respect to a nuclear receptor and a CHIP are retained. Specific examples of other proteins or peptides to be fused are as given above.

A CHIP and a nuclear receptor not bound to a nuclear receptor ligand are bound together via a chaperone protein. In other words, the chaperone protein binds to the nuclear receptor not bound to a nuclear receptor ligand, and the CHIP binds to the chaperone protein bound to the nuclear receptor not bound to a nuclear receptor ligand, thus binding the CHIP to the nuclear receptor not bound to a nuclear receptor ligand via the chaperone protein. Binding of the CHIP to the nuclear receptor not bound to a nuclear receptor ligand via the chaperone protein is nuclear receptor ligand-independent (does not require the presence of a nuclear receptor ligand). Moreover, the nuclear receptor not bound to a nuclear receptor ligand is ubiquitinated by the CHIP bound to the nuclear receptor not bound to a nuclear receptor ligand via the chaperone protein, and the nuclear receptor not bound to a nuclear receptor ligand is nuclear receptor ligand-independently degraded.

A gene encoding a CHIP or a chaperone protein comprises an open reading frame encoding a CHIP or chaperone protein, and a termination codon located at the 3′ end thereof. A gene encoding a CHIP or chaperone protein may also comprise an untranslated region (UTR) at the 5′ end and/or 3′ end of the open reading frame. Moreover, a gene encoding a CHIP or chaperone protein may also comprise the open reading frame of another protein or peptide such that a CHIP or chaperone protein is expressed as a fused protein.

An example of a gene encoding a wild CHIP is a gene comprising DNA consisting of the sequence of nucleotides 57 through 965 in the nucleotide sequence represented by SEQ ID NO:1. The sequence of nucleotides 57 through 965 in the nucleotide sequence represented by SEQ ID NO:1 is an open reading frame encoding a wild CHIP, and in the nucleotide sequence represented by SEQ ID NO:1, the translation initiation codon is located at the sequence of nucleotides 57 through 59, while the termination codon is located at the sequence of nucleotide 966 through 968. There are no particular limits on the nucleotide sequence of a gene encoding a wild CHIP as long as the wild CHIP is encoded, and the nucleotide sequence of the open reading frame is not limited to the sequence of nucleotides 57 through 965 in the nucleotide sequence represented by SEQ ID NO:1.

An example of a gene encoding a wild Hsc70 is a gene comprising DNA consisting of the sequence of nucleotides 79 through 2016 in the nucleotide sequence represented by SEQ ID NO:3. The sequence of nucleotides 79 through 2016 in the nucleotide sequence represented by SEQ ID NO:3 is an open reading frame encoding a wild Hsc70, and in the nucleotide sequence represented by SEQ ID NO:3, the translation initiation codon is located at the sequence of nucleotides 79 through 81, while the termination codon is located at the sequence of nucleotides 2017 through 2019. There are no particular limits on the nucleotide sequence of a gene encoding a wild Hsc70 as long the wild Hsc70 is encoded, and the nucleotide sequence of the open reading frame is not limited to the sequence of nucleotides 79 through 2016 in the nucleotide sequence represented by SEQ ID NO:3.

An example of a gene encoding a wild Hsp70 is a gene comprising DNA consisting of the sequence of nucleotides 178 through 2100 in the nucleotide sequence represented by SEQ ID NO:5. The sequence of nucleotides 178 through 2100 in the nucleotide sequence represented by SEQ ID NO:5 is an open reading frame encoding a wild Hsp70, and in the nucleotide sequence represented by SEQ ID NO:5, the translation initiation codon is located at the sequence of nucleotides 178 through 180 while the termination codon is located at the sequence of nucleotides 2101 through 2103. There are no particular limits on the nucleotide sequence of a gene encoding a wild Hsp70 as long as the wild Hsp70 is encoded, and the nucleotide sequence of the open reading frame is not limited to the sequence of nucleotides 178 through 2100 in the nucleotide sequence represented by SEQ ID NO:5.

An example of a gene encoding a wild Hsp90 is a gene comprising DNA consisting of the sequence of nucleotides 98 through 2269 in the nucleotide sequence represented by SEQ ID NO:7. The sequence of nucleotides 98 through 2269 in the nucleotide sequence represented by SEQ ID NO:7 is an open reading frame encoding a wild Hsp90, and in the nucleotide sequence represented by SEQ ID NO:7, the translation initiation codon is located at the sequence of nucleotides 98 through 100, while the termination codon is located at the sequence of nucleotides 2270 through 2272. There are no particular limits on the nucleotide sequence of a gene encoding a wild Hsp90 as long as the wild Hsp90 is encoded, and the nucleotide sequence of the open reading frame is not limited to the sequence of nucleotides 98 through 2269 in the nucleotide sequence represented by SEQ ID NO:7.

An example of a gene encoding a mutant CHIP is a gene comprising DNA which hybridizes under stringent conditions with DNA complementary to DNA consisting of the sequence of nucleotides 57 through 965 in the nucleotide sequence represented by SEQ ID NO:1, and which encodes a protein capable of binding to a chaperone protein.

“Stringent conditions” are for example conditions of 42° C., 2×SSC and 0.1% SDS or preferably 65° C., 0.1×SSC and 0.1% SDS, and an example of DNA which hybridizes under stringent conditions with DNA complementary to DNA consisting of the sequence of nucleotides 57 through 965 in the nucleotide sequence represented by SEQ ID NO:1 is DNA having 50% or greater or preferably 65% or greater or more preferably 80% or greater homology with DNA consisting of the sequence of nucleotides 57 through 965 in the nucleotide sequence represented by SEQ ID NO:1.

An example of a gene encoding a mutant Hsc70 is a gene comprising DNA which hybridizes under stringent conditions with DNA complementary to DNA consisting of the sequence of nucleotides 79 through 2016 in the nucleotide sequence represented by SEQ ID NO:3, and which encodes a protein capable of binding to a nuclear receptor and a CHIP.

“Stringent conditions” are for example conditions similar to those described above, and an example of DNA which hybridizes under stringent conditions with DNA complementary to DNA consisting of the sequence of nucleotides 79 through 2016 in the nucleotide sequence represented by SEQ ID NO:3 is DNA having 50% or greater or preferably 65% or greater or more preferably 80% or greater homology with DNA consisting of the sequence of nucleotides 79 through 2016 in the nucleotide sequence represented by SEQ ID NO:3.

An example of a gene encoding a mutant Hsp70 is a gene comprising DNA which hybridizes under stringent conditions with DNA complementary to DNA consisting of the sequence of nucleotides 178 through 2100 in the nucleotide sequence represented by SEQ ID NO:5, and which encodes a protein capable of binding to a nuclear receptor and a CHIP.

“Stringent conditions” are for example conditions similar to those described above, and an example of DNA which hybridizes under stringent conditions with DNA complementary to DNA consisting of the sequence of nucleotides 178 through 2100 in the nucleotide sequence represented by SEQ ID NO:5 is DNA having 50% or greater or preferably 65% or greater or more preferably 80% or greater homology with DNA consisting of the sequence of nucleotides 178 through 2100 in the nucleotide sequence represented by SEQ ID NO:5.

An example of a gene encoding a mutant Hsp90 is a gene comprising DNA which hybridizes under stringent conditions with DNA complementary to DNA consisting of the sequence of nucleotides 98 through 2269 in the nucleotide sequence represented by SEQ ID NO:7, and which encodes a protein capable of binding to a nuclear receptor and a CHIP.

“Stringent conditions” are for example conditions similar to those described above, and an example of DNA which hybridizes under stringent conditions with DNA complementary to DNA consisting of the sequence of nucleotides 98 through 2269 in the nucleotide sequence represented by SEQ ID NO:7 is DNA having 50% or greater or preferably 65% or greater or more preferably 80% or greater homology with DNA consisting of the sequence of nucleotides 98 through 2269 in the nucleotide sequence represented by SEQ ID NO:7.

Judging whether a substance can suppress or promote binding of a CHIP with a nuclear receptor via a chaperone protein can be accomplished for example as follows, but the judgment method is not limited thereby.

A CHIP, a chaperone protein and a nuclear receptor are brought into contact with each other in the presence or absence of a test substance, the amount of binding of the CHIP with the nuclear receptor is measured, and the amount of binding in the presence of the test substance is compared to the amount of binding in the absence the test substance. If the results show that the amount of binding in the presence of the test substance is less than the amount of binding in the absence of the test substance, the test substance can be judged to be capable of suppressing binding of the CHIP with the nuclear receptor via the chaperone protein. If the amount of binding in the presence of the test substance is more than the amount of binding in the absence of the test substance, the test substance can be judged to be capable of promoting binding of the CHIP with the nuclear receptor via the chaperone protein.

Since degradation of a nuclear receptor in a cell is though to progress through binding of a CHIP with a nuclear receptor via a chaperone protein (for example, degradation of an estrogen receptor in a cell progresses through binding of a CHIP with an estrogen receptor via a Hsc70), a substance capable of suppressing binding of a CHIP with a nuclear receptor via a chaperone protein may have the action of suppressing degradation of a nuclear receptor, while a substance capable of promoting binding of a CHIP with a nuclear receptor via a chaperone protein may have the action of promoting degradation of a nuclear receptor. Consequently, a substance which can suppress binding of a CHIP with a nuclear receptor via a chaperone protein can be screened as a substance which can suppress degradation of a nuclear receptor, while a substance which can promote binding of a CHIP with a nuclear receptor via a chaperone protein can be screened as a substance which can promote degradation of a nuclear receptor.

A substance which can suppress or promote binding of a CHIP with a nuclear receptor via a chaperone protein may be a substance which acts on one of CHIP, chaperone protein and nuclear receptor, or it may be a substance which acts on two or more of them. A substance which can suppress binding of a CHIP with a nuclear receptor via a chaperone protein may be a substance which can suppress binding of a CHIP in a dissociated state with a chaperone protein bound to a nuclear receptor or not bound to a nuclear receptor, a substance which can dissociate a CHIP in a bound state from a chaperone protein bound to a nuclear receptor or not bound to a nuclear receptor, a substance which can suppress binding of a chaperone protein in a dissociated state with a nuclear receptor, or a substance which can dissociate a chaperone protein in a bound state from a nuclear receptor.

A CHIP, a chaperone protein and a nuclear receptor are brought into contact with each other in vitro or in vivo.

When contact is in vitro, (i) endogenous proteins extracted from cells or tissues which express the target proteins, (ii) recombinant proteins extracted from cultures of transformants prepared by introduction into host cells of recombinant vectors capable of expressing the target proteins, or (iii) chemically synthesized peptides or the like can be used as the CHIP, the chaperone protein and the nuclear receptor.

When contact is in vivo, (i) endogenous proteins present in cells or (ii) recombinant proteins present in transformants prepared by introduction into host cells of recombinant vectors capable of expressing the target proteins or the like can be used as the CHIP, the chaperone protein and the nuclear receptor.

When a CHIP, a chaperone protein and a nuclear receptor are brought into contact with each other, a wild CHIP, a mutant CHIP, a derivative of a wild CHIP or mutant CHIP, or a fused protein of a wild CHIP or mutant CHIP with another protein or peptide or the like can be used as the CHIP. The same is true of the chaperone protein and the nuclear receptor.

When a CHIP, a chaperone protein and a nuclear receptor are brought into contact with each other, conditions which affect binding of a CHIP with a nuclear receptor via a chaperone protein are adjusted so that the binding is dependent on the presence or absence of a test substance.

Conditions which affect binding of a CHIP with a nuclear receptor via a chaperone protein include for example temperature, type of solvent, concentration of CHIP, concentration of chaperone protein, concentration of nuclear receptor, presence or absence of intervening proteins which modulate binding of a CHIP with a nuclear receptor via a chaperone protein (that is, proteins which directly or indirectly affect binding of the three and promote, inhibit or regulate the binding) and the like.

The temperature when a CHIP, a chaperone protein and a nuclear receptor are brought into contact with each other can be set for example at 0 to 50° C. A solvent such as a buffer of pH 5.5 to 8.5 containing univalent or bivalent salts, or a buffer with hydrophobicity adjusted by addition of ethanol, ethylene glycol, glycerin, DMSO or the like can be used as the solvent when a CHIP, a chaperone protein and a nuclear receptor are brought into contact with each other. The concentration of CHIP, chaperone protein or nuclear receptor can be set for example at 0.1 μg/ml to 100 μg/ml. The intervening protein may be for example a protein which competes with a CHIP by binding to the same region of chaperone protein to which the CHIP binds, a protein which controls binding of a CHIP with a chaperone protein by means of phosphorylation or another modification, a co-activator or co-repressor which competes with a chaperone protein by binding to the same region of nuclear receptor to which the chaperone protein binds, or a protein which controls binding of a chaperone protein with a nuclear receptor by means of phosphorylation or another modification.

When binding of a CHIP with a nuclear receptor via a chaperone protein is independent on a nuclear receptor ligand, there is no need to include a nuclear receptor ligand, but a nuclear receptor ligand may be included.

The amount of binding of a CHIP with a nuclear receptor via a chaperone protein is measured for example using as the marker the amount of bound units formed by binding of the CHIP with the nuclear receptor, or the volume of a signal produced by binding of the CHIP with the nuclear receptor or the like.

The amount of bound units formed by binding of a CHIP with a nuclear receptor can be measured for example by adding a label substance to one or more of the CHIP, the chaperone protein and the nuclear receptor, bringing the CHIP, the chaperone protein and the nuclear receptor into contact with each other, isolating the bound units, and measuring using the amount of labeled substance on the bound units as the marker. Specifically, it can be measured as follows using the GST pull down method. RI is added to either the CHIP or the nuclear receptor while the other is made into a fused protein with GST, the CHIP, the chaperone protein and the nuclear receptor are brought into contact with each other, and the bound units are adsorbed by a glutathione sepharose column. The column is washed, and proteins binding to the column are eluted. The eluted proteins are subjected to SDS-PAGE to isolate the bound units, and the amount of bound units is measured using the amount of RI on the bound units as the marker.

In addition, the amount of bound units formed by binding of a CHIP with a nuclear receptor can be measured by well-known protein analysis techniques, such as Western blotting using antibodies or fragments thereof capable of the bound units, immune precipitation, ELISA, tissue immunoblotting or the like. “Antibodies” include either monoclonal antibodies or polyclonal antibodies, and “monoclonal antibodies” and “polyclonal antibodies” include all classes of monoclonal antibodies and polyclonal antibodies. “Fragments of antibodies” include Fab fragments, F(ab)′₂ fragments, single-chain antibodies (scFv) and the like.

There are no particular limits on the kind of signal generated by binding of a CHIP with a nuclear receptor, and examples include expression of a reporter gene, fluorescence resonance energy transfer (FRET), surface plasmon resonance (SPR) and detection of local density changes by movement of the frequency of a quartz oscillator and the like.

When the signal generated by binding of a CHIP with a nuclear receptor via a chaperone protein is expression of a reporter gene, the amount of binding of the CHIP with the nuclear receptor via the chaperone protein can be measured for example as follows using the transcriptional activation function of the nuclear receptor.

An expression vector comprising a gene encoding a CHIP, an expression vector comprising a gene encoding a chaperone protein, an expression vector comprising a gene encoding a nuclear receptor and an expression unit comprising a reporter gene and a nuclear receptor-dependent promoter attached upstream from the reporter gene are introduced into host cells, and the amount of reporter activity produced by expression of the reporter gene is measured. The promoter selected for attachment upstream from the reporter gene is one whose degree of activation by the nuclear receptor is altered by binding of the CHIP with the nuclear receptor via the chaperone protein. Thus, when the CHIP binds with the nuclear receptor via the chaperone protein, the degree of promoter activation by the nuclear receptor changes, and the amount of expression of the reporter gene changes. In other words, the changes in amount of reporter activity produced by expression of the reporter gene are a marker for amount of binding of the CHIP with the nuclear receptor via the chaperone protein.

The reporter gene may be for example the beta-galactosidase gene, chloramphenicol acetyltransferase gene, luciferase gene, ampicillin resistance gene, tetracycline resistance gene, kanamycin resistance gene or the like, and the reporter activity may be for example beta-galactosidase activity, chloramphenicol acetyltransferase activity, luciferase activity, ampicillin resistance, tetracycline resistance, kanamycin resistance or the like.

When the signal generated by binding of a CHIP with a nuclear receptor via a chaperone protein is fluorescence resonance energy transfer (FRET), the amount of binding of the CHIP with the nuclear. receptor via the chaperone protein can be measured for example as follows.

Either the CHIP or the nuclear receptor is fused with a fluorescent protein (donor) while the other is fused with a fluorescent protein (acceptor), the CHIP, the chaperone protein and the nuclear receptor are brought into contact with each other, and the amount of fluorescence generated by binding of the CHIP with the nuclear receptor via the chaperone protein is measured. In this case, CFP (cyan fluorescent protein) can be used as the fluorescent protein (donor), and YFP (yellow fluorescent protein) as the fluorescent protein (acceptor). Fluorescence resonance energy transfer (FRET) is a phenomenon in which excitation energy is transferred from the fluorescence group of one molecule (donor) to the fluorescence group of another molecule (acceptor), and the excited acceptor emits the energy as heat or new fluorescence. Since the two molecules need to be somewhat close together for fluorescence resonance energy transfer (FRET) to operate efficiently, it us useful as an effective means of detecting interactions between proteins within cells.

Judging whether a test substance can suppress or promote binding of a CHIP with a chaperone protein can be accomplished for example as follows, but the judgment method is not limited thereby.

A CHIP and a chaperone protein are brought into contact in the presence or absence of a test substance, the amount of binding of the CHIP with the chaperone protein is measured, and the amount of binding in the presence of the test substance is compared with the amount of binding in the absence of the test substance. If in the results the amount of binding in the presence of the test substance is less than the amount of binding in the absence of the test substance, the test substance can be judged to be capable of suppressing binding of the CHIP with the chaperone protein. If the amount of binding in the presence of the test substance is greater than the amount of binding without the presence of the test substance, the test substance can be judged to be capable of promoting the binding of the CHIP with the chaperone protein.

Since degradation of a nuclear receptor in a cell is thought to proceed through binding of a CHIP with a nuclear receptor via a chaperone protein (that is, binding of a CHIP with a chaperone protein and binding of a chaperone protein with a nuclear receptor), a substance which can suppress binding of a CHIP with a chaperone protein may have the action of suppressing degradation of a nuclear receptor, while a substance which can promote binding of a CHIP with a chaperone protein may have the action of promoting degradation of a nuclear receptor. Consequently, a substance which can suppress binding of a CHIP with a chaperone protein can be screened as a substance which can suppress degradation of a nuclear receptor, and a substance which can promote binding of a CHIP with a chaperone protein can be screened as a substance which can promote degradation of a nuclear receptor.

A substance which can suppress or promote binding of a CHIP with a chaperone protein may be either a substance which acts on either of CHIP and chaperone protein, or a substance which acts on both. Moreover, a substance which can suppress binding of a CHIP with a chaperone protein may be either a substance which can suppress binding of a CHIP in a dissociated state with a chaperone protein bound to a nuclear receptor or not bound to a nuclear receptor, or a substance which can dissociate a CHIP in a bound state from a chaperone protein bound to a nuclear receptor or not bound to a nuclear receptor.

A CHIP and a chaperone protein can be brought into contact either in vitro or in vivo.

When contact is in vitro, (i) endogenous proteins extracted from cells or tissues which express the target proteins, (ii) recombinant proteins extracted from cultures of transformants prepared by introduction into host cells of recombinant vectors capable of expressing the target proteins, or (iii) chemically synthesized peptides or the like can be used as the CHIP and the chaperone protein.

When contact is in vivo, (i) endogenous proteins present in cells or (ii) recombinant proteins present in transformants prepared by introduction into host cells of recombinant vectors capable of expressing the target proteins or the like can be used as the CHIP and the chaperone protein.

When bringing a CHIP into contact with a chaperone protein, a wild CHIP, a mutant CHIP, a derivative of a wild CHIP or mutant CHIP, or a fused protein of a CHIP with another protein or peptide or the like can be used as the CHIP. The same is true of the chaperone protein.

When bringing a CHIP into contact with a chaperone protein, conditions which affect binding of the two are adjusted so that binding of the two is dependent on the presence or absence of the test substance.

Conditions which affect binding of a CHIP with a chaperone protein include for example temperature, type of solvent, concentration of CHIP, concentration of chaperone protein, presence or absence of intervening proteins which modulate binding of a CHIP with a chaperone protein (that is, proteins which directly or indirectly affect binding of the two and promote, inhibit or regulate the binding) and the like.

The temperature when bringing a CHIP into contact with a chaperone protein can be set for example at 0 to 50° C. A solvent such as a buffer of pH 5.5 to 8.5 containing univalent or bivalent salts, or a buffer with hydrophobicity adjusted by addition of ethanol, ethylene glycol, glycerin, DMSO or the like can be used as the solvent for bringing a CHIP into contact with a chaperone protein. The concentration of CHIP, the concentration of chaperone protein can be set for example at 0.1 μg/ml to 100 μg/ml. The intervening protein may be for example a protein which competes with a CHIP by binding to the same region of chaperone protein to which the CHIP binds, or a protein which controls binding of a CHIP with a chaperone protein by means of phosphorylation or another modification.

The amount of binding of a CHIP with a chaperone protein can be measured in the same way as the amount of binding of a CHIP with a nuclear receptor via a chaperone protein.

Judging whether a test substance can suppress or promote binding of a chaperone protein with a nuclear receptor can be accomplished for example as follows, but the judgment method is not limited thereby.

A chaperone protein and a nuclear receptor are brought into contact in the presence or absence of a test substance, the amount of binding of the chaperone protein with the nuclear receptor is measured, and the amount of binding in the presence of the test substance is compared with the amount of binding in the absence of the test substance. If in the results the amount of binding in the presence of the test substance is less than the amount of binding in the absence of the test substance, the test substance can be judged to be capable of suppressing binding of the chaperone protein with the nuclear receptor. If the amount of binding in the presence of the test substance is greater than the amount of binding without the presence of the test substance, the test substance can be judged to be capable of promoting the binding of the chaperone protein with the nuclear receptor.

Since degradation of a nuclear receptor in a cell is thought to proceed through binding of a CHIP with a nuclear receptor via a chaperone protein (that is, binding of a CHIP with a chaperone protein and binding of a chaperone protein with a nuclear receptor), a substance which can suppress binding of a chaperone protein with a nuclear receptor may have the action of suppressing degradation of a nuclear receptor, while a substance which can promote binding of a chaperone protein with a nuclear receptor may have the action of promoting degradation of a nuclear receptor. Consequently, a substance which can suppress binding of a chaperone protein with a nuclear receptor can be screened as a substance which can suppress degradation of a nuclear receptor, and a substance which can promote binding of a chaperone protein with a nuclear receptor can be screened as a substance which can promote degradation of a nuclear receptor.

A substance which can suppress or promote binding of a chaperone protein with a nuclear receptor may be either a substance which acts on either of chaperone protein and nuclear receptor, or a substance which acts on both. Moreover, a substance which can suppress binding of a chaperone protein with a nuclear receptor may be either a substance which can suppress binding of the chaperone protein with the nuclear receptor dissociated from the chaperone protein, or a substance which can dissociate the chaperone protein bound to the nuclear receptor from the nuclear receptor.

A chaperone protein and a nuclear receptor can be brought into contact either in vitro or in vivo.

When contact is in vitro, (i) endogenous proteins extracted from cells or tissues which express the target proteins, (ii) recombinant proteins extracted from cultures of transformants prepared by introduction into host cells of recombinant vectors capable of expressing the target proteins, or (iii) chemically synthesized peptides or the like can be used as the chaperone protein and the nuclear receptor.

When contact is in vivo, (i) endogenous proteins present in cells or (ii) recombinant proteins present in transformants prepared by introduction into host cells of recombinant vectors capable of expressing the target proteins or the like can be used as the chaperone protein and the nuclear receptor.

When bringing a chaperone protein into contact with a nuclear receptor, a wild chaperone protein, a mutant chaperone protein, a derivative of a wild chaperone protein or mutant chaperone protein, or a fused protein of a chaperone protein with another protein or peptide or the like can be used as the chaperone protein. The same is true of the nuclear receptor.

When bringing a chaperone protein into contact with a nuclear receptor, conditions which affect binding of the two are adjusted so that binding of the two is dependent on the presence or absence of the test substance.

Conditions which affect binding of a chaperone protein with a nuclear receptor include for example temperature, type of solvent, concentration of chaperone protein, concentration of nuclear receptor, presence or absence of intervening proteins which modulate binding of a chaperone protein with a nuclear receptor (that is, proteins which directly or indirectly affect binding of the two and promote, inhibit or regulate the binding) and the like.

The temperature when bringing a chaperone protein into contact with a nuclear receptor can be set for example at 0 to 50° C. A solvent such as a buffer of pH 5.5 to 8.5 containing univalent or bivalent salts, or a buffer with hydrophobicity adjusted by addition of ethanol, ethylene glycol, glycerin, DMSO or the like can be used as the solvent for bringing a chaperone protein into contact with a nuclear receptor. The concentration of chaperone protein and the concentration of nuclear receptor can be set for example at 0.1 μg/ml to 100 μg/ml. The intervening protein may be for example a co-activator or co-repressor which competes with a chaperone protein by binding to the same region of nuclear receptor to which the chaperone protein binds, or a protein which controls binding of a chaperone protein with a nuclear receptor by means of phosphorylation or another modification.

The amount of binding of a chaperone protein with a nuclear receptor can be measured in the same way as the amount of binding of a CHIP with a nuclear receptor via a chaperone protein.

Judging whether a test substance can suppress or promote ubiquitination of a nuclear receptor by CHIP can be accomplished for example as follows, but the judgment method is not limited thereby.

A CHIP bound to a nuclear receptor via a chaperone protein and ubiquitin are reacted in the presence or absence of a test substance, the amount of ubiquitination of the nuclear receptor by CHIP is measured, and the amount of ubiquitination in the presence of the test substance is compared with the amount of ubiquitination in the absence of the test substance. If in the results the amount of ubiquitination in the presence of the test substance is less than the amount of ubiquitination in the absence of the test substance, the test substance can be judged to be capable of suppressing ubiquitination of the nuclear receptor by CHIP. If the amount of ubiquitination in the presence of the test substance is more than the amount of ubiquitination in the absence of the test substance, the test substance can be judged to be capable of promoting ubiquitination of the nuclear receptor by CHIP.

Since degradation of a nuclear receptor in a cell is thought to proceed through ubiquitination of a nuclear receptor by CHIP (for example, degradation of an estrogen receptor in a cell proceeds through ubiquitination of an estrogen receptor by CHIP), a substance which can suppress ubiquitination of a nuclear receptor by CHIP may have the action of suppressing degradation of a nuclear receptor, while a substance which can promote ubiquitination of a nuclear receptor by CHIP may have the action of promoting degradation of a nuclear receptor. Consequently, a substance which can suppress ubiquitination of a nuclear receptor by CHIP can be screened as a substance which can suppress degradation of a nuclear receptor, and a substance which can promote ubiquitination of a nuclear receptor by CHIP can be screened as a substance which can promote degradation of a nuclear receptor.

A substance which can suppress or promote ubiquitination of a nuclear receptor by CHIP may be either a substance which acts on any one of CHIP, nuclear receptor and ubiquitin, or a substance which acts on two or more of these. A substance which can suppress ubiquitination of a nuclear receptor by CHIP include a substance which can suppress the ability of ubiquitination of CHIP, and a substance which can suppress binding of a nuclear receptor with ubiquitin.

Reaction with ubiquitin may be conducted either in vitro or in vivo.

When reaction is in vitro, (i) endogenous proteins extracted from cells or tissues which express the target proteins, (ii) recombinant proteins extracted from cultures of transformants prepared by introduction into host cells of recombinant vectors capable of expressing the target proteins, or (iii) chemically synthesized peptides or the like can be used as the CHIP, the nuclear receptor, the chaperone protein and the ubiquitin.

When reaction is in vivo, (i) endogenous proteins present in cells or (ii) recombinant proteins present in transformants prepared by introduction into host cells of recombinant vectors capable of expressing the target proteins or the like can be used as the CHIP, the nuclear receptor, the chaperone protein and the ubiquitin.

When reacting in vivo or in vitro, a wild CHIP, a mutant CHIP, a derivative of a wild CHIP or mutant CHIP or a fused protein of a CHIP with another protein or peptide can be used as the CHIP. The same is true of the chaperone protein, the nuclear receptor and the ubiquitin.

When reacting in vivo or in vitro, conditions which affect ubiquitination of the nuclear receptor by CHIP are adjusted so that ubiquitination of the nuclear receptor by CHIP is dependent on the presence or absence of the test substance.

Conditions which affect ubiquitination of a nuclear receptor by CHIP include for example temperature, type of solvent, presence or absence of ubiquitin transfer proteins E1 and E2, concentration of CHIP, concentration of chaperone protein, concentration of ubiquitin, concentration of nuclear receptor, concentrations of E1 and E2, type of E2, type of nuclear receptor, presence or absence and type of post-translation modification of the nuclear receptor and the like. Ubiquitination of a nuclear receptor by CHIP requires ubiquitin transfer proteins E1 (ubiquitin-activating enzyme) and E2 (ubiquitin-conjugating enzyme). Consequently, E1 and E2 are included when ubiquitination of a nuclear receptor by CHIP is conducted.

The reaction temperature can be set for example at 0 to 50° C. A solvent such as a buffer of pH 5.5 to 8.5 containing univalent or bivalent salts, or a buffer with hydrophobicity adjusted by addition of ethanol, ethylene glycol, glycerin, DMSO or the like can be used as the reaction solvent. The concentration of CHIP, the concentration of chaperone protein, the concentration of ubiquitin, the concentration of nuclear receptor and the concentrations of E1 and E2 can be set at 0.1 μg/ml to 100 μg/ml for example. Examples of post-translation modifications of the nuclear receptor include post-translation modification by phosphate groups, methyl groups, acetyl groups or the like.

The amount of ubiquitination of a nuclear receptor by CHIP can be measured for example using the amount of binding between nuclear receptor and ubiquitin as the marker. The amount of binding of the nuclear receptor and ubiquitin can be measured in the same way as the amount of binding of a CHIP with a nuclear receptor via a chaperone protein for example.

Judging whether a test substance can suppress or promote expression of a gene encoding a CHIP or chaperone protein can be accomplished for example as follows, but the judgment method is not limited thereby.

In vivo, for example, a test substance is administered to a model animal which express a gene encoding a CHIP or chaperone protein, a specimen (cells or tissues in which a gene encoding a CHIP or chaperone protein is expressed) is collected from the model animal, the level of expression of the gene in the specimen is measured, and the level of expression before administration of the test substance is compared with the level of expression after administration of the test substance. If in the results the level of expression after administration of the test substance is lower than the level of expression before administration of the test substance, the test substance can be judged to be capable of suppressing expression of the gene. On the other hand, if the level of expression after administration of the test substance is higher than the level of expression before administration of the test substance, the test substance can be judged to be capable of promoting expression of the gene.

A mammal other than human (such as a cow, sheep, goat, horse, pig, rabbit, dog, cat, rat, mouse or the like) which is a transgenic animal made to artificially express a gene encoding a CHIP or chaperone protein can be used as the model animal. The transgenic animal can be prepared by a well-known method, such as (i) a method of mixing a gene encoding a CHIP or chaperone protein with an egg and treating them with calcium phosphate, (ii) a method of directly introducing a gene encoding a CHIP or chaperone protein into the nucleus of an egg at the pronucleus stage under phase contrast microscopy (micro-injection), (iii) a method using embryonic stem cells (ES cells) or the like.

In vitro, for example, a test substance is first brought into contact with a specimen (tissue or cells in which a gene encoding a CHIP or chaperone protein is expressed), the level of expression of the gene in the specimen is measured, and the level of expression before administration of the test substance is compared with the level of expression after administration of the test substance. If in the results the level of expression after administration of the test substance is smaller than the level of expression before administration of the test substance, the test substance can be judged capable of suppressing expression of the gene. On the other hand, if the level of expression after administration of the test substance is greater than the level of expression before administration of the test substance, the test substance can be judged capable of promoting expression of the gene.

A cell strain derived from humans, monkeys, mice, rats or the like or cells made to artificially express a gene encoding a CHIP or chaperone protein for example can be used as the cells with which the test substance is brought into contact. Such cells can be prepared by inserting a gene encoding a CHIP or chaperone protein into a suitable expression vector, and introducing that vector into suitable host cells.

Since degradation of a nuclear receptor in cells is thought to proceed through binding of a CHIP with a nuclear receptor via a chaperone protein, a substance which can suppress expression of a gene encoding a CHIP or chaperone protein may have the action of suppressing degradation of a nuclear receptor, while a substances which can promote expression of a gene encoding a CHIP or chaperone protein may have the action of promoting degradation of a nuclear receptor. Consequently, a substance which can suppress expression of a gene encoding a CHIP or chaperone protein can be screened as a substance which can suppress degradation of a nuclear receptor, while a substance which can promote expression of a gene encoding a CHIP or chaperone protein can be screened as a substance which can promote degradation of a nuclear receptor.

A substance which can suppress or promote expression of a gene encoding a CHIP or a chaperone protein may be either a substance which can suppress or promote either the transcription process or the translation process of the gene, or a substance which can suppress or promote both.

The level of expression of a gene encoding a CHIP or chaperone protein includes the level of transcription of the gene into mRNA and the level of translation into a protein. Consequently, the level of expression of a gene encoding a CHIP or chaperone protein can be measured based on the existing amount of mRNA or the existing amount of the protein.

A well-known gene analysis technique such as a hybridization (such as the Northern hybridization method, dot blot method, DNA micro-array method or the like) or gene amplification technique (such as RT-PCR or the like) can be used for measuring the existing amount of mRNA.

When using a hybridization technique, an oligonucleotide or polynucleotide capable of hybridizing with mRNA, cDNA or the like encoding a CHIP or chaperone protein can be used as a probe, while when using a gene amplification technique, such an oligonucleotide or polynucleotide can be used as a primer. The nucleotides making up the oligonucleotide or polynucleotide may be either deoxyribonucleotides or ribonucleotides. There are no particular limits on the nucleotide length of the oligonucleotide, which is normally 7 to 100 nucleotides or preferably 15 to 50 nucleotides. There are also no particular limits on the nucleotide length of the polynucleotide, which is normally 30 to 1500 nucleotides or preferably 50 to 1000 nucleotides.

The oligonucleotide or polynucleotide should preferably be capable of hybridizing specifically with mRNA, cDNA or the like encoding a CHIP or chaperone protein. “Capable of hybridizing specifically” signifies the ability to hybridize under stringent conditions, and “stringent conditions” are for example conditions of 42° C., 2×SSC and 0.1% SDS, or preferably 65° C., 0.1×SSC and 0.1% SDS.

The nucleotide sequence of an oligonucleotide or polynucleotide capable of hybridizing with mRNA, cDNA or the like encoding a CHIP or chaperone protein can be designed based on the nucleotide sequence of nucleic acids encoding the CHIP or chaperone protein. A restriction enzyme recognition sequence, tag or the like can be added to the 5′ end of the primer, and a fluorescent dye, radioisotope or other label can be added to the primer and probe.

A specific method of measuring the existing amount of mRNA encoding a CHIP or chaperone protein is explained with RT-PCR used as an example. Total RNA is extracted from a specimen, cDNA is synthesized from the extracted total RNA, PCR is performed using a primer capable of hybridizing with cDNA encoding a CHIP or chaperone protein and using the synthesized cDNA as the template, and the PCR amplified fragments are assayed in order to measure the existing amount of mRNA encoding the CHIP or chaperone protein. PCR in this case is performed under conditions such that the produced amount of PCR amplified fragments reflects the amount of the initial template cDNA (for example, the number of PCR cycles at which the PCR amplified fragments increase exponentially).

There are no particular limits on the method of assaying the PCR amplified fragments, which can be assayed for example using an assay method employing radioisotopes (RI) or one employing a fluorescent dye, for example.

Examples of assay methods employing RI include for example (i) a method in which a RI-labeled nucleotide (such as ³²P labeled dCTP or the like) is added to the reaction liquid as a substrate and incorporated into the PCR amplified fragments to RI label the PCR amplified fragments, the PCR amplified fragments are isolated by electrophoresis or the like, and the PCR amplified fragments are assayed by measuring radioactivity, (ii) a method in which the PCR amplified fragments are RI-labeled through the use of an RI-labeled primer, the PCR amplified fragments are isolated by electrophoresis or the like, and the PCR amplified fragments are assayed by measuring radioactivity, and (iii) a method in which the PCR amplified fragments are subjected to electrophoresis, blotted on a membrane and hybridized with an RI-labeled probe, and the PCR amplified fragments are assayed by measuring radioactivity. Radioactivity can be measured for example using a liquid scintillation counter, x-ray film, imaging plate or the like.

Examples of assay methods employing fluorescent dyes include (i) a method in which PCR amplification fragments are dyed using a fluorescent dye (such as ethidium bromide (EtBr), SYBR Green I, PicoGreen or the like) which intercalates with double-stranded DNA, and the PCR amplification fragments are assayed by measuring the strength of fluorescence produced by exposure to excitation light, or (ii) a method in which PCR amplification fragments are labeled with fluorescent dye through the use of a primer labeled with the fluorescent dye, the PCR amplification fragments are isolated by electrophoresis, and the PCR amplification fragments are assayed by measuring the fluorescent strength. Fluorescent strength can be measured for example using a CCD camera, fluorescence scanner, fluorescence spectrophotometer or the like.

A well-known protein analysis technique can be used for measuring the existing amount of CHIP or chaperone protein, such as Western blotting using antibodies or fragments thereof capable of reacting to a CHIP or chaperone protein, immune precipitation, ELISA, tissue immunoblotting or the like.

When using antibodies or fragments thereof capable of reacting to a CHIP or chaperone protein to measure the existing amount of CHIP or chaperone protein, a method such as radio-immunoassay (RIA), enzyme immunoassay (EIA), chemical luminescence immunoassay (CLIA), fluorescence immunoassay (FIA), tissue immunoassay or the like can be used. Specifically, a CHIP or chaperone protein in a specimen is first captured using a solid-phase carrier (such as an immunoplate, latex particles or the like) to which the antibodies have been bound by physical adsorption, chemical binding or the like, and the captured CHIP or chaperone protein can then be assayed using labeled antibodies (such as antibodies labeled with an enzyme such as peroxidase or alkaline phosphatase, a fluorescent substance such as fluorescein, umbelliferone or the like) having an antigen recognition site for the CHIP or chaperone protein different from that of the antibodies fixed on the solid-phase carrier.

Measurement of the existing amount of CHIP or chaperone protein can be accomplished by measuring the activity of CHIP or chaperone protein. The activity of CHIP or chaperone protein can be measured for example by a well-known method such as Western blotting, ELISA or the like using antibodies or fragments thereof capable of reacting to a CHIP or chaperone protein.

It is preferable that measurement values for the level of expression of a gene encoding a CHIP or chaperone protein be corrected based on measurement values for the level of expression of a gene encoding a protein (such a beta-actin or GAPDH) the expressed level of which does not fluctuate greatly.

A recombinant vector capable of expressing a target protein (such as CHIP, chaperone protein, nuclear receptor, ubiquitin or the like) can be prepared for example by preparing a DNA fragment of a suitable length which comprises the coding region of the target protein, and then inserting the DNA fragment downstream from the promoter of a suitable expression vector. The nucleotide sequence of the coding region of the target protein should preferably have nucleotides substituted so as to achieve the optimal codon for expression in the host cells. The DNA fragment needs to be incorporated into the vector so that its functions are expressed, and in addition to the promoter the vector may contain enhancers and other cis elements, splicing signals, poly A addition signals, selection markers (such as the dihydrofolate reductase gene, ampicillin resistance gene or neomycin resistance gene), ribosome binding sequences (SD sequences) and the like.

There are no particular limits on the expression vector as long as it is capable of self-sustained replication in the host cells, and for example a plasmid vector, phage vector, virus vector or the like may be used. Examples of plasmid vectors include E. coli derived plasmids (such as pRSET, pBR322, pBR325, pUC118, pUC119, pUC18 and pUC19), B. subtilis derived plasmids (such as pUB110 and pTP5) and yeast derived plasmids (such as YEp13, YEp24 and YCp50), examples of phage vectors include gamma-phages (such as Charon4A, Charon21A, EMBL3, EMBL4, gamma gt10, gamma gt11 and gamma ZAP), and examples of virus vectors include retrovirus, vaccinia virus, adenovirus and other animal viruses and baculovirus and other insect viruses.

Hosts cells such as prokaryotic cells, yeasts, animal cells, insect cells, plant cells and the like can be used for introduction of the recombinant vector. Individual animals, plants, silkworms or the like can also be used.

Bacteria which can be used as the host cells include for example Escherichia coli and other Escherichia, Bacillus subtilis and other Bacillus, Pseudomonas putida and other Pseudomonas, and Rhizobium meliloti and other Rhizobium. Specific examples include Escherichia coli including Escherichia coli XL1-Blue, Escherichia coli XL2-Blue, Escherichia coli DH1, Escherichia coli K12, Escherichia coli JM109 and Escherichia coli HB101 or Bacillus subtilis including Bacillus subtilis MI114 and Bacillus subtilis 207-21 and the like. There are no particular limits on the promoter in this case as long as it is capable of expression in E. coli and other bacteria, and for example a promoter such as a trp promoter, lac promoter, P_{L} promoter, P_{R} promoter or other promoter derived from E. coli or phages or an artificially designed and modified promoter such as a tac promoter, lacT7 promoter, let I promoter or the like can be used.

There are no particular limits on the method of introducing the recombinant vector into bacteria as long as it is a method capable of introducing DNA into bacteria, and for example electroporation or a method employing calcium ions or the like can be used.

Yeasts which can be used as host cells include for example Saccharomyces cerevisiae, Schizosaccharomyces pombe, Pichia pastoris and the like. There are no particular limits on the promoter in this case as long as it is capable of expression in yeasts, and for example a gal1 promoter, gal10 promoter, heat shock protein promoter, MFα1 promoter, PHO5 promoter, PGK promoter, GAP promoter, ADH promoter, AOX1 promoter or the like can be used.

There are no particular limits on the method of introducing the recombinant vector into a yeast as long as it is a method capable of introducing DNA into yeasts, and for example the electroporation method, spheroplast method, lithium acetate method or the like can be used.

Animal cells which can be used as host cells include for example monkey COS-7 cells, Vero, Chinese hamster ovarian cells (CHO cells), mouse L cells, rat GH3, human FL cells and the like. There are no particular limits on the promoter in this case as long as it is capable of expression in animal cells, and for example an SRα promoter, SV40 promoter, LTR (long terminal repeat) promoter, CMV promoter, human cytomegalovirus early gene promoter or the like can be used.

There are no particular limits on the method of introducing the recombinant vector into animal cells as long as it is a method capable of introducing DNA into animal cells, and for example the electroporation method, calcium phosphate method or lipofection method can be used.

Insect cells which can be used as host cells include for example ovarian cells of Spodoptera frugiperda, ovarian cells of Trichoplusia ni, cultured cells derived from silkworm ovaries and the like. Examples of Spodoptera frugiperda ovarian cells include Sf9, Sf21 and the like, examples of Trichoplusia ni ovarian cells include High 5, BTI-TN-5B 1-4 (Invitrogen) and the like, and examples of cultured cells derived from silkworm ovaries include Bombyx mori N4 and the like.

There are no particular limits on the method of introducing the recombinant vector into insect cells as it is a method capable of introducing DNA into insect cells, and for example the calcium phosphate method, lipofection method, electroporation method or the like can be used.

Culturing of a transformant prepared by introduction of a recombinant vector into host cells can be accomplished by ordinary methods used for culturing host cells.

The medium used for culturing a transformant obtained with E. coli or a yeast or other microorganism as the host cells may contain carbon sources, nitrogen sources, inorganic salts or the like which can be recovered by the microorganism, and may be either a natural or synthetic medium as long as it is one in which culture of the transformant can be accomplished efficiently. Carbon sources which can be used include glucose, lactose, sucrose, starch and other carbohydrates, acetic acid, propionic acid and other organic acids, and ethanol, propanol and other alcohols. Nitrogen sources which can be used include ammonia, ammonium chloride, ammonium sulfate, ammonium acetate, ammonium phosphate and other ammonium salts of inorganic or organic acids, and peptone, meat extract, yeast extract, corn steep liquor, casein hydrolysate and the like. Inorganic salts which can be used include monopotassium phosphate, dipotassium phosphate, magnesium phosphate, magnesium sulfate, sodium chloride, ferrous sulfate, manganese sulfate, copper sulfate, calcium carbonate and the like.

Culturing of the transformant can be accomplished under aerobic conditions such as shaking culture, aerated spinner incubation or the like. The culture temperature is normally 30 to 37° C., the culture time is normally 12 to 16 hours, and the pH during culture is maintained at 6.0 to 8.0. pH can be adjusted using inorganic acids, organic acids, alkaline solutions, urea, calcium carbonate, ammonia or the like. Moreover, an antibiotic such as ampicillin, tetracycline or the like can be added to the medium as necessary for purposes of culture.

When culturing a microorganism transformed with an expression vector using an inducible promoter as the promoter, an inducer can be added to the medium as necessary. For example, isopropyl-β-D-thiogalactopyranoside or the like can be added to the medium when culturing a microorganism transformed with an expression vector using a lac promoter, and indoleacrylic acid or the like when culturing a microorganism transformed with an expression vector using a trp promoter.

Media which can be used for culturing a transformant obtained using animal cells as the host cells include commonly used RPMI1640 medium, Eagle's MEM medium, α-MEM medium, DMEM medium and these media with fetal calf serum or the like added thereto. Culture of the transformant is normally accomplished at 30 to 37° C. for 1 to 7 days under 5% CO₂. Moreover, an antibiotic such as kanamycin, penicillin, streptomycin, neomycin, hygromycin, blasticidin or the like may be added to the medium as necessary for purposes of culture.

A medium such as commonly used TNM-FH medium (Pharmingen), TC-100 medium (Gibco BRL), Sf-900 II SFM medium (Gibco BRL), ExCell400, ExCell 405 (JRH Biosciences) or the like can be used as the medium for culturing a transformant obtained with insect cells as the host cells. The transformant is normally cultured at 25 to 28° C. for 48 to 96 hours. An antibiotic such as gentamicin or the like can be added to the medium as necessary for purposes of culture.

The target protein can be extracted from the culture of the transformant. The term “culture” here includes culture supernatant, cultured cells, cultured bacterial bodies and crushed cells or bacterial bodies. When the target protein accumulates in cells of the transformant, cells in the culture can be collected by centrifuging the culture, after which the cells are washed and crushed and the target protein is extracted. When the target protein is excreted outside the cells of the transformant, the target protein can be extracted from the culture supernatant. The extracted protein can be purified using a method such as solvent extraction, salting-out/desalinization with ammonium sulfate, precipitation with an organic solvent, diethyl-amino-ethyl (DEAE)-sepharose, ion exchange chromatography, hydrophobic chromatography, gel filtration, affinity chromatography or the like.

The target protein can be chemically synthesized using the Fmoc (fluorenyl methyloxycarbonyl) method, tBoc (t-butyloxycarbonyl) method or the like. In this case a commercial peptide synthesizer can be used.

The target protein can be synthesized using an in vitro transcription and translation system. An in vitro transcription and translation system consists of an in vitro transcription system capable of transcribing mRNA from nucleic acids in vitro and an in vitro translation system capable of translating proteins from mRNA in vitro, and an in vitro transcription and translation system includes all the elements (such as polymerase, ribosomes, tRNA and the like) necessary for transcription and translation. A specific example of an in vitro transcription and translation system is a cell-free transcription and translation system prepared from an extract of eukaryotic and prokaryotic cells, and specific examples of cell-free transcription and translation systems include cell-free transcription and translation systems prepared from cell extracts of E. coli (such as E. coli S30), wheat germ, rabbit reticulocytes, mouse L cells, Ehrlich's ascites carcinoma cells, HeLa cells, CHO cells, budding yeasts and the like.

A target gene (such as a gene encoding a CHIP, a gene encoding a chaperone protein, a gene encoding a nuclear receptor or a gene encoding a ubiquitin) can be obtained for example by preparing a cDNA library and screening the cDNA library for clones comprising the target DNA.

To prepare a cDNA library, total RNA is first obtained from cells or tissues of mammals (such as humans, monkeys, cows, sheep, goats, horses, pigs, rabbits, dogs, cats, mice, rats or the like), and poly(A)+RNA (mRNA) is obtained by the batch method or affinity method using oligo dT-cellulose, poly U-sepharose or the like. In this case poly(A)+RNA (mRNA) can also be fractioned by sucrose density-gradient centrifugation. Next, single-stranded cDNA is synthesized using oligo dT primer and reverse transcriptase with the resulting mRNA as the template, and then double-stranded cDNA is synthesized from this single-stranded cDNA. The resulting double-stranded cDNA is incorporated into a suitable cloning vector to prepare a recombinant vector which is used to transform E. coli or other host cells, and the transformants are selected using tetracycline resistance or ampicillin resistance as the marker to obtain a cDNA library. The cloning vector for preparing the cDNA library may be any capable of self-sustained replication in the host cells, and for example a phage vector, plasmid vector or the like can be used. E. coli or the like for example can be used as the host cells.

A commercial kit such as the SuperScript Plasmid System for cDNA Synthesis and Plasmid Cloning (Gibco BRL) or ZAP-cDNA Synthesis Kit (Stratagene) can be used in preparing the cDNA library.

To screen clones comprising the target DNA from the cDNA library, the primer is synthesized based on the nucleotide sequence of the target gene and used to perform a polymerase chain reaction (PCR) from which PCR amplified fragments are obtained. The PCR amplified fragments can also be sub-cloned using a suitable plasmid vector.

The target DNA can be obtained by colony hybridization or plaque hybridization of the cDNA library using the PCR amplified fragments as the probe. The PCR amplified fragments labeled with an isotope (such as ³²P or ³⁵S), biotin, digoxigenin or the like can be used as the probe. Clones containing the target DNA can be obtained by an expression screening method such as immunoscreening using antibodies or the like.

The nucleotide sequence of the resulting DNA can be determined by inserting the DNA fragments into a vector by ordinary methods, either as is or after cleavage with a suitable restriction enzyme, and applying a commonly used method of nucleotide sequence analysis such as the Maxam-Gilbert chemical modification method or dideoxynucleotide chain termination method, but normally the sequence is determined using a 373A DNA sequencer (Perkin Elmer) or other nucleotide sequence analysis device.

The target gene can be chemically synthesized according to its nucleotide sequence. Chemical DNA synthesis can be accomplished using a commercial DNA synthesizer such as a DNA synthesizer using the thiophosphite method (Shimazu Manufacturing) or a DNA synthesizer using the phosphoramidite method (Perkin Elmer), for example.

If the target gene is a gene encoding a mutant CHIP, mutant chaperone protein or mutant nuclear receptor, a well-known method such as site-directed mutagenesis or the like for example can be used for artificially introducing mutations into a gene encoding a wild CHIP, wild chaperone protein or wild nuclear receptor. For example, a Mutant-K (Takara), Mutant-G (Takara) or LA PCR in vitro Mutagenesis series kit (Takara) can be used.

Antibodies capable of reacting to the target protein can be prepared using the target protein as the immunizing antigen.

To prepare polyclonal antibodies, rats, mice, guinea pigs, rabbits, sheep, horses, cows or other mammals are immunized using the immunizing antigen. Immunization is performed multiple times following emulsification with an immune enhancer such as Freund's complete adjuvant in order to induce antibody production. Immune enhancers which can be used include Freund's complete adjuvant (FCA), Freund's incomplete adjuvant (FIA), aluminum hydroxide gel and the like. The dose of antigen per individual mammal is set according to the species of mammal. Administration may be intravenous, subcutaneous or intraperitoneal, for example. Several days after the last immunization, antibody titer for the target protein is measured, blood is collected after antibody titer has risen, and antiserum is obtained. Antibody titer can be measured by a method such as enzyme-linked immunosorbent assay (ELISA), radio-immuno-assay (RIA) or the like.

When antibodies need to be purified from the antiserum, a well-known method such as salting out with ammonium sulfate, gel chromatography, ion exchange chromatography, affinity chromatography or the like or a combination of these can be selected as appropriate.

To prepare monoclonal antibodies, mammals are immunized with an immunizing antigen as in the case of the polyclonal antibodies, and antibody-producing cells are collected several days after the final immunization. Examples of antibody-producing cells include spleen cells, lymphocytes, thymocytes, peripheral blood cells and the like, but spleen cells are ordinarily used.

Next, cell fusion of the antibody-producing cells with myeloma cells is performed to obtain a hybridoma. Myeloma cells which can be fused with the antibody-producing cells are cells derived from humans, mice or other mammals which are commonly available cell strains. The cell strain used is preferably one which has drug selectivity and which has the property of being unable to survive in selection medium (such as HAT medium) in an unfused state and only being able to survive when fused with antibody-producing cells. Specific examples of myeloma cells include P3X63-Ag.8.U1 (P3U1), P3/NSI/1-Ag4-1, Sp2/0-Ag14 and other mouse myeloma cell strains.

Cell fusion is accomplished by mixing specific proportions of the antibody-producing cells and myeloma cells in an animal cell culture medium such as DMEM, RPMI-1640 or the like which does not contain serum, and then fusing them in the presence of a cell fusion promoter such as polyethylene glycol or by electrical pulse treatment (for example, electroporation).

Following cell fusion treatment, culture is performed in selection medium to select the target hybridoma. The culture supernatant of the multiplied hybridoma is screened to determine whether the target antibodies are present. Ordinary methods can be used to screen the hybridoma without any particular limitations. For example, some of the culture supernatant contained in a well grown as a hybridoma can be collected, and screened by enzyme-linked immunosorbent assay (ELISA), radio-immuno-assay (RIA) or the like.

The hybridoma can be cloned for example by limiting dilution analysis, the soft agar method or fibrin gel method, fluorescent excitation cell sorting or the like, to ultimately obtain a hybridoma which produces monoclonal antibodies. A common method of cell culture or the like can be used as the method of obtaining monoclonal antibodies from the resulting hybridoma.

When the monoclonal antibodies need to be purified, a well-known method such as salting out with ammonium sulfate, gel chromatography, ion exchange chromatography, affinity chromatography or the like or a combination of these can be selected as appropriate.

A substance screened by the present invention which can suppress or promote degradation of a nuclear receptor is a substance which can suppress or promote nuclear receptor ligand-independent degradation of a nuclear receptor not bound to a nuclear receptor ligand, and a substance screened by the present invention which can suppress or promote degradation of a nuclear receptor is useful in the prevention or treatment of osteoporosis, obesity, diabetes, hyperlipidemia, menopausal disorders, hormone-dependent cancers and other hormonal diseases and the like.

### EXAMPLES

In the following test examples, cell culture, construction of recombinant vectors, transient transfection, preparation of cell nucleus extracts, GST pull-down, immune precipitation and Western blotting were performed as follows.

(1) Cell Culture

HeLa cells, human breast cancer-derived MCF-7 cells and human fetal kidney-derived 293 cells were cultured at 37° C. in Dulbecco's modified Eagle's medium (DMEM) containing 10% FCS in humidified air containing 5% (partial pressure) carbon dioxide gas.

(2) Construction of Recombinant Vectors

Full-length cDNA encoding CHIP (SEQ ID NO:1) was inserted into pCDNA3 (Invitrogen) to construct a recombinant vector. In addition, cDNA encoding ERα or deletion mutants of ERα was inserted into pCDNA3 to construct recombinant vectors.

(3) Transient Transfection

The target DNA was diluted with 100 μL of OptiMEM medium (Life Technologies, Inc.). The amount of DNA was adjusted to a total of 1 μg by addition of blank vector. 10 μL of lipofectamine reagent (Life Technologies, Inc.) was diluted in the same way with 100 μL of OptiMEM medium. After being left at room temperature for 5 minutes, the two liquids were mixed and left for an additional 20 minutes to form a complex. This mixture was then dripped directly onto cells seeded on 12-well plates and cultured to 40-50% confluence in DMEM containing 0.2% FCS treated with activated charcoal, and transfection was performed. 6 hours after transfection normal medium was substituted, and after 24 hours' further culture the ligand was added.

(4) Preparation of Cell Nucleus Extracts

Preparation of cell nucleus extracts was accomplished by well-known methods (Yanagisawa, J. et al, Molecular Cell (2002) March, 9(3):553-562), with modifications.

(5) GST Pull-Down and Immune Precipitation

GST pull-down was performed by well-known methods (Yanagisawa, J. et al, Molecular Cell (2002) March, 9(3):553-562). About 50 μg of MCF-7 cell nucleus extract or 293 cell nucleus extract was incubated for 1 hour at 4° C. in 25 mM tris-buffered saline containing 0.1% Tween20 (T-TBS) together with 50 μg of either glutathione sepharose beads or anti-Flag tag antibody beads (Santa Cruz Biotechnology). Beads to which protein had bound were washed 4 times with 25 mM tris-buffered saline containing 0.1% Tween20 and centrifuged for 1 minute at 5000 rpm, and the beads were collected. Protein was eluted from the beads by boiling in loading buffer.

(6) Western Blotting

Western blotting using the respective antibodies was accomplished by well-known methods (Yanagisawa, J. et al, Molecular Cell (2002) March, 9(3):553-562) with some alterations. 10 μg of each extract of MCF-7 cells and 293 cells was isolated by 8% or 10% SDS-PAGE, and transferred to a nitrocellulose membrane. This membrane was incubated for 1 hour in a blocking solution of 1% BSA (bovine serum albumin) and 5% skim milk added to 25 mM tris-buffered saline containing 0.02% Tween20 (T-TBS), and then reacted with the respective antibodies for 1 hour at 25° C. After being washed 3 times for 10 minutes each in 25° C. T-TBS, the membranes were incubated for 1 minute at 25° C. with peroxidase-bound goat anti-mouse antibodies. Protein that reacted with the antibodies was made visible with an Amersham Pharmacia high-sensitivity chemical luminescence detection system (ECL).

### Test Example 1

### Degradation of Estrogen Receptor Alpha (ERα) in the Absence of Estrogen

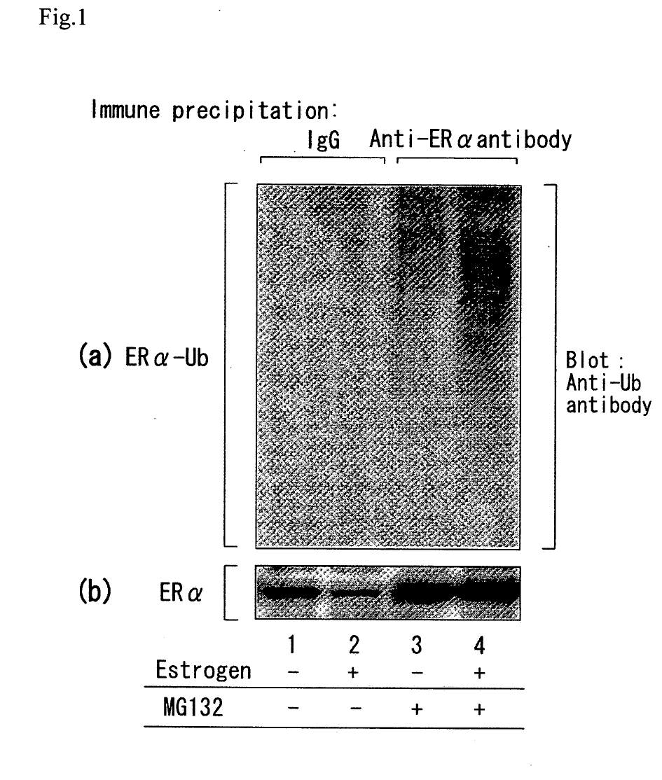

With the object of investigating ubiquitination and degradation of estrogen receptor alpha (ERα) in the presence and absence of estrogen, MCF-7 cells were cultured for 24 hours in the presence and absence of 10 nM estrogen, nucleus extracts were prepared, and the amount of ERα and the amount of ubiquitination in the extracts was measured. When the nucleus extracts were developed by SDS-PAGE and Western blotting was performed using anti-ERα antibodies, the amount of ERα fell dramatically when estrogen was added to the medium (see FIG. 1(b), lane 2). This indicates that the MCF-7 cells are provided with an estrogen-dependent ERα degradation mechanism. However, when the proteasome inhibitor MG132 was added to the medium at a concentration of 1 μM, the amount of ERα increased dramatically both when estrogen was added to the medium (see FIG. 1(b), lane 4) and when estrogen was not added to the medium (see FIG. 1(b), lane 3). This suggests that MCF-7 cells are provided with a mechanism that also degrades ERα estrogen-independently.

With the aim of confirming whether this degradation is due to ubiquitination, ERα was first purified from MCF-7 cell nucleus extract by immune precipitation and developed by SDS-PAGE, Western blotting was performed with anti-ubiquitin antibodies (anti-Ub antibodies), and the amount of ubiquitination of ERα was measured. In the results, ubiquitinated ERα (ERα-Ub) was detected both when estrogen was added to the medium (see FIG. 1(a), lane 4) and when estrogen was not added to the medium (see FIG. 1(a), lane 3).

The results above point to the existence of a hitherto unknown estrogen-independent ERα degradation mechanism.

### Test Example 2

### Test Showing that ERαΔAD Also Undergoes Estrogen-Independent Degradation

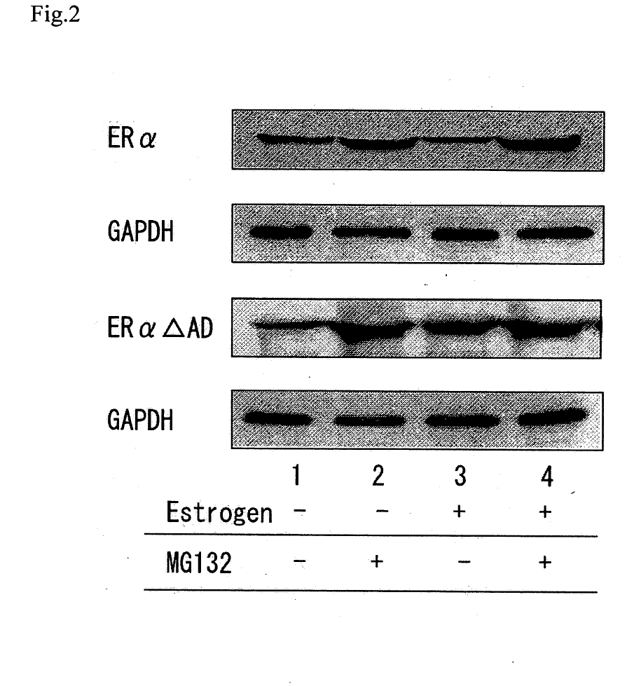

With the aim of investigating the mechanisms of estrogen-dependent and estrogen-independent degradation of ERα, an ERα deletion mutant was used to investigate whether the two types of degradation are driven by the same mechanism. It has become clear from the inventors' research that the amino acids beginning with amino acid 535 of ERα are required for binding with factors involved in degradation of ERα. ERαΔAD, which lacks the part of the amino acid sequence beginning with amino acid 535 of ERα (helix 12 part), was therefor used as a deletion mutant of ERα. ERαΔAD binds to ligands, but the structural changes required for transcriptional activation are not induced by binding of the ligands. ERαΔAD was made to be expressed in 293 cells, and degradation was measured in the presence and absence of 10 nM of estrogen. In the results, it was shown that ERαΔAD does not undergo estrogen-dependent degradation (see FIG. 2, lane 3), but does undergo estrogen-independent degradation (see FIG. 2, lane 1).

This degradation is inhibited by addition of 1 μM of MG132 (proteasome inhibitor) as in the case of full-length ERα (see FIG. 2, lanes 2 and 4), showing that it is caused by the proteasome.

The above results indicate that the estrogen-dependent degradation and estrogen-independent degradation of ERα are promoted by different molecular mechanisms.

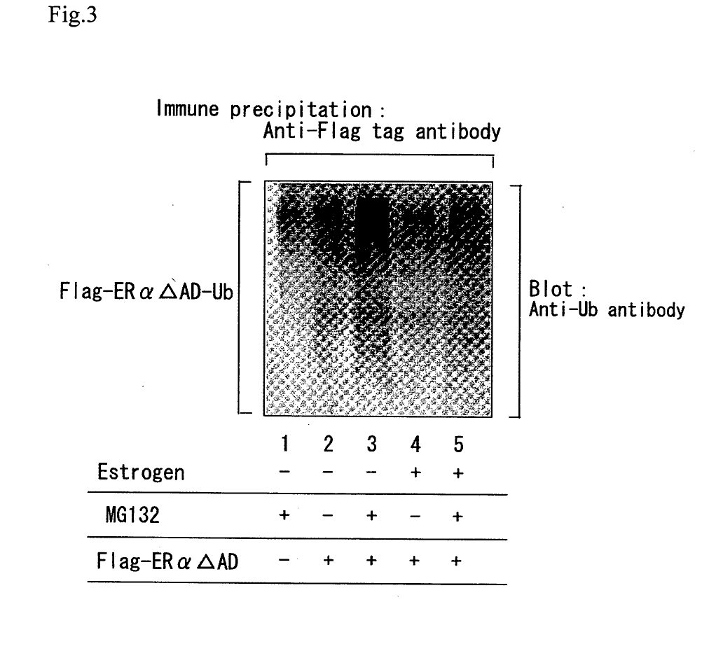

The reactivity to MG132 suggested that the estrogen-independent degradation of ERαΔAD also follows a pathway from ubiquitination to the proteasome, but ubiquitination of ERαΔAD was detected for purposes of confirmation. A Flag tag-ERαΔAD fused protein (Flag-ERαΔAD) was made to be expressed in 293 cells, and after culturing in the presence of estrogen and MG132 a nucleus extract was prepared, immune precipitation was performed with anti-Flag tag antibodies, the collected protein was developed by SDS-polyacrylamide gel electrophoresis, and ubiquitinated ERαΔAD (Flag-ERαΔAD-Ub) was detected by Western blotting using anti-ubiquitin antibodies (anti-Ub antibodies). In the results, strong accumulation of a polyubiquitin protein was observed when no estrogen was added but MG132 was added (see FIG. 3, lane 3). When estrogen was added but no MG132 was added (see FIG. 3, lane 4), the signal obtained was about the same as the background.

The results above show that as in the case of full-length ERα, estrogen-independent degradation of ERαΔAD occurs by polyubiquitination followed by degradation by the proteasome.

### Test Example 3

### Determination of Region Required for Estrogen-Independent Degradation of ERα

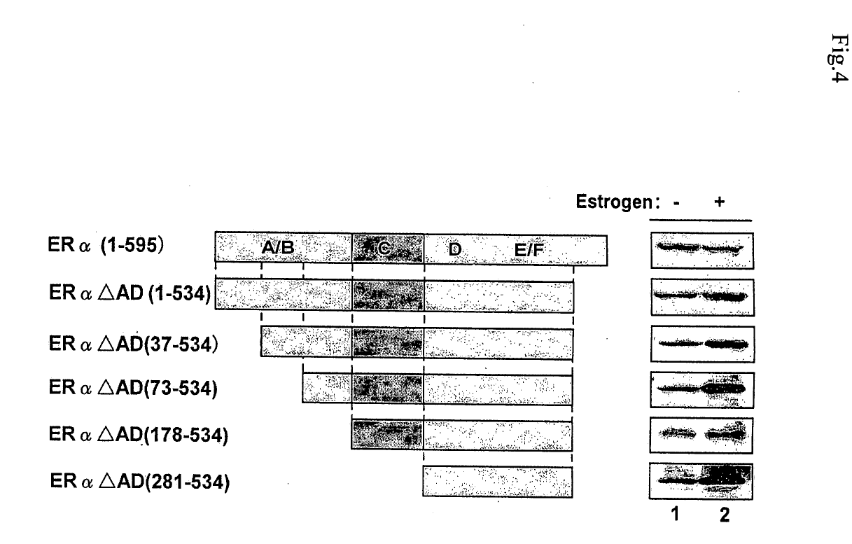

When the aim of analyzing the estrogen-independent degradation of ERα in more detail, analysis was performed to determine what domain of ERα is necessary for degradation. Systematic deletion mutant genes of ERα were prepared so that Flag tag fused proteins were expressed, and transfected into 293 cells, and degradation in the presence and absence of estrogen was detected by Western blotting using anti-Flag tag antibodies. As shown in FIG. 4, ERαΔAD (1-534) consisting of amino acids 1 through 534, ERαΔAD (37-534) consisting of amino acids 37 through 534, ERαΔAD (73-534) consisting of amino acids 73 through 534, ERαΔAD (178-534) consisting of amino acids 178 through 534, and ERαΔAD (281-534) consisting of amino acids 281 through 534 were used as the ERα deletion mutants. In the results, each form of ERαΔAD, a mutant which does not undergo estrogen-dependent degradation, underwent estrogen-independent degradation (see FIG. 4 lane 1). Moreover, ligand-independent degradation was observed even when the sequence from the N terminus to amino acid 280 of ERα was deleted (see FIG. 4, lane 1), showing that the part of ERα required for ligand-independent degradation is located between amino acids 281 and 534.

### Test Example 4

### Identification of a Protein Which Binds Estrogen-Independently to ERα

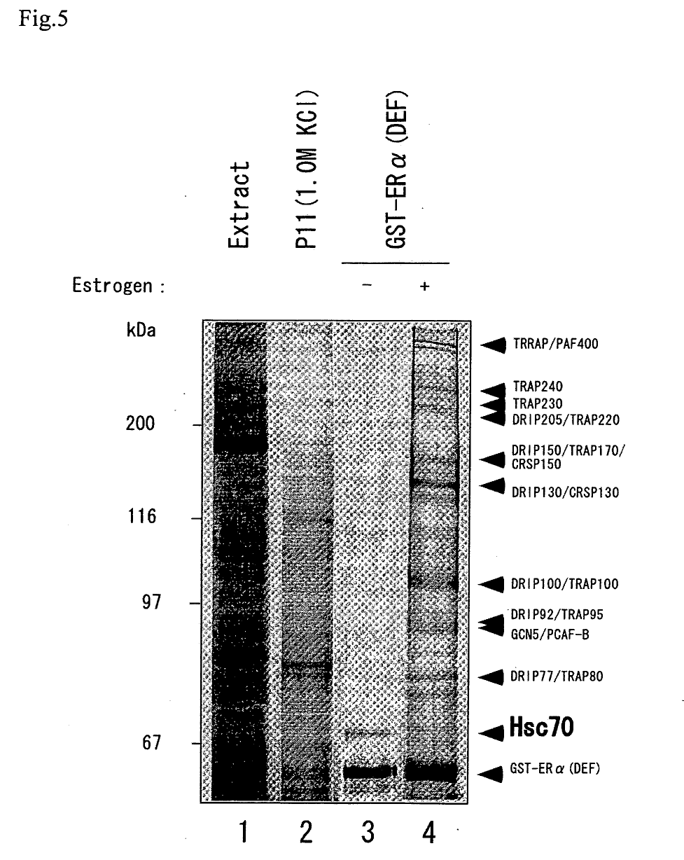

A protein which binds estrogen-independently to an estrogen receptor was isolated and purified from HeLa cell raw nucleus extract. Following reported methods (Yanagisawa, J. et al, Molecular Cell 2002 March, 9(3):553-62), a protein fraction eluted from a phosphocellulose column with buffer containing 1.0 M potassium chloride was added to a column (balanced with buffer containing 100 mM KCl) of glutathione sepharose beads (Amersham-Pharmacia) fixed with a fused protein of GST and the ligand-binding domain of ERα, and washing and elution were performed. The eluted fraction was developed by 7.5% SDS-polyacrylamide gel electrophoresis and CBB stained, after which the various bands were separated (see FIG. 5). Protein was eluted from the separated bands, and following trypsin degradation an MS fingerprint was obtained with a MALDI-TOF mass spectrometer (Perceptive Co. Voyager). When the resulting fingerprint was searched on a public database (University of California), it matched Hsc70, a known heat shock protein. Since CHIP (carboxyl-terminus of Hsc70 interacting protein), a protein having the characteristic U-box of ubiquitin ligases, is known to bind to Hsc70 (Meacham, G C et al, “The Hsc70 co-chaperone CHIP targets immature CFTR for preteasomal degradation,” Nat. Cell Biol. 3, 100-105, 2001), it was hypothesized that CHIP is involved in the estrogen-independent degradation of ERα.

### Test Example 5

### Binding of ERα and CHIP in the Absence of Estrogen

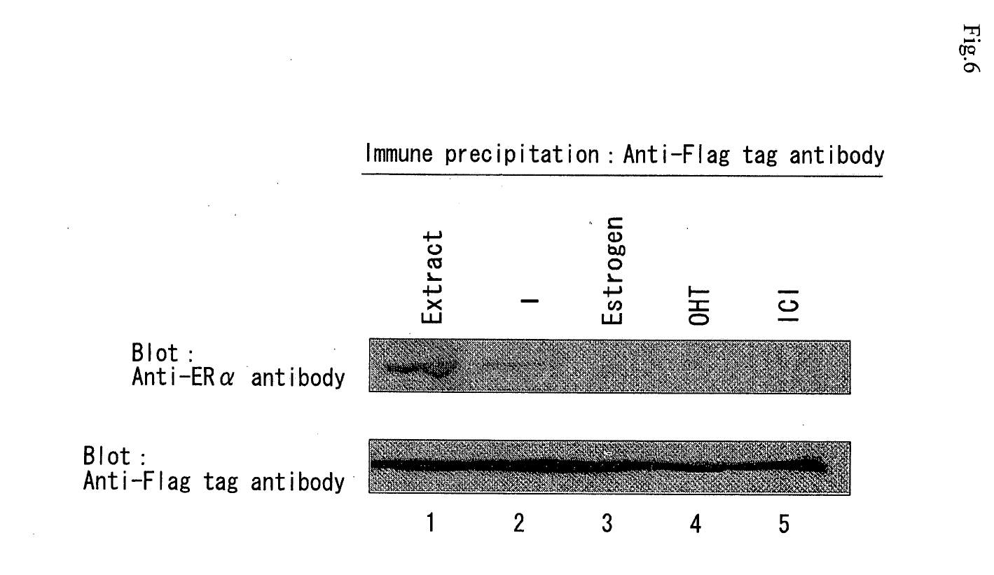

An immune precipitation test was performed with the aim of proving that CHIP binds ligand-independently to ERα. The ERα gene and a Flag tag-CHIP fused protein gene were transiently transfected into 293 cells and cultured for 24 hours without addition of a ligand or with addition of 10 nM estrogen, 1 μM tamoxifen (OHT) or 100 nM fulvestrant (ICI) as ligands, and the protein complexes bound with CHIP were immune precipitated from the nucleus extracts with anti-Flag tag antibodies. The collected protein fractions were developed by SDS-polyacrylamide gel electrophoresis, and Western blotting was performed with anti-ERα antibodies or anti-Flag tag antibodies to detect the proteins. In the results, ERα was co-precipitated with CHIP only when cultured without the addition of a ligand (see FIG. 6, lane 2), showing that CHIP binds ligand-independently to ERα.

### Test Example 6

### Promotion of Estrogen-Independent Degradation of ERα by CHIP

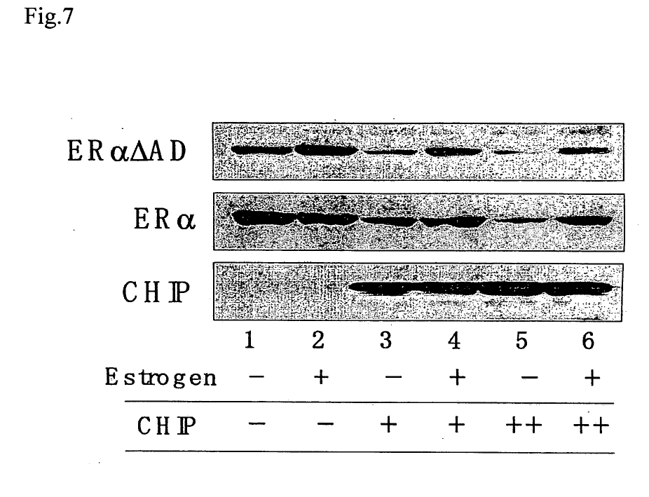

In order to confirm that CHIP actually promotes degradation of ERα, 293 cells which strongly expressed ERαΔAD, ERα and CHIP, respectively, were cultured in the presence and absence of estrogen, nucleus extracts were prepared, and the amount of degradation of ERαΔAD and ERα was measured by Western blotting. In the results, both ERαΔAD and ERα were strongly degraded when estrogen was not added (see FIG. 7, lane 3). This degradation increased when the expressed amount of CHIP was increased (see FIG. 7, lane 5), suggesting that ligand-independent degradation of ERα is performed by CHIP.

### Test Example 7

### Test Showing that CHIP does not Promote Degradation of ERα Bound to Artificial ERα Ligand

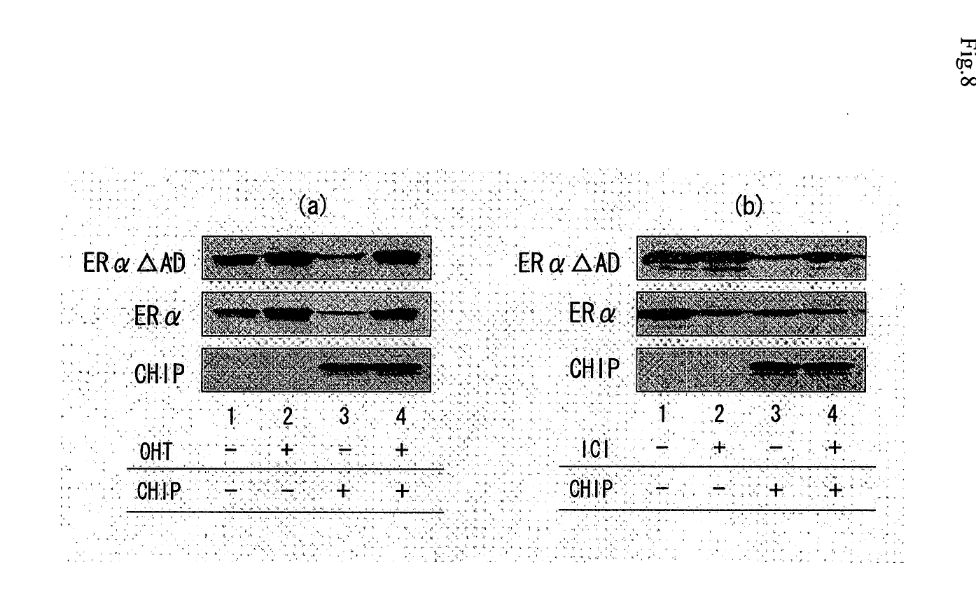

It was already shown that CHIP does not bind to or degrade ERα having bound estrogen, but a test was done to confirm that it does not degrade ERα bound to the artificial ERα ligands tamoxifen (OHT) and fulvestrant (ICI). As shown in FIG. 6, it was already shown that CHIP does not bind to ERα having bound OHT or ICI. As in FIG. 6, 293 cells which strongly expressed ERαΔAD, ERα and CHIP, respectively, were cultured for 24 hours in the presence or absence of 1 μM OHT or 100 nM ICI, nucleus extracts were prepared, and the amount of degradation of ERαΔAD and ERα was measured by Western blotting. As in the case of estrogen, degradation of ERα was only detected when OHT or ICI was not added (See FIGS. 8(a) and (b), lane 3). These results show that while unlike estrogen, OHT and ICI do not transcriptionally activate ERα, like estrogen they do block degradation of ERα by CHIP. OHT and ICI are used as therapeutic drugs for estrogen-dependent cancers because they block transcriptional activation by blocking interaction of ERα with transcriptional co-factors, but they are similar to estrogen in their interactions with CHIP.

### Test Example 8

### Test Showing that Estrogen-independent Degradation of ERα by CHIP is a Quality-control Mechanism for Intracellular ERα

CHIP or CHIPΔUbox (a mutant having the ubiquitin ligase activity removed) were made to be expressed in cells already co-expressing normal ERα and a dominant negative mutant of ERα, and the transcriptional activation ability of ERα was measured. That is, four kinds of plasmids, (1) a plasmid comprising the ERα gene, (2) a plasmid comprising a dominant negative mutant (one of four) of the ERα gene, (3) a plasmid comprising the CHIP gene or CHIPΔUbox gene and (4) a reporter plasmid having the luciferase gene attached downstream from the estrogen response element (ERE), were transiently introduced into 293 cells, and the transcriptional activation ability of ERα when estrogen was added was measured using luciferase activity as the marker. After introduction of the plasmid comprising the CHIP gene or CHIPΔUbox gene into the 293 cells they were cultured for 48 hours in the absence of estrogen so that degradation of ERα would progress sufficiently. Luciferase activity was measured in cells collected after having been cultured for 12 hours following administration of estrogen. The amount of ERα in the cell nucleus extract was measured at the same time by Western blotting. ERα(V364E), ERα(C447A), ERαΔAD and ERα(L540Q) were used as the dominant negative mutants of ERα. ERα(V364E) and ERα(C447A) are temperature sensitive mutants having mutations in the ligand-binding domain, and their transcriptional activity is low at the normal body temperature of 37° C. because the protein does not take its normal folded form. ERαΔAD is a helix 12 deletion mutant, while ERα(L540Q) is a helix 12 single-residue substitution mutant, and the transcriptional activity of both is low because they can no longer bind to transcriptional activation factors. ERαΔAD and ERα(L540Q) are not temperature sensitive.

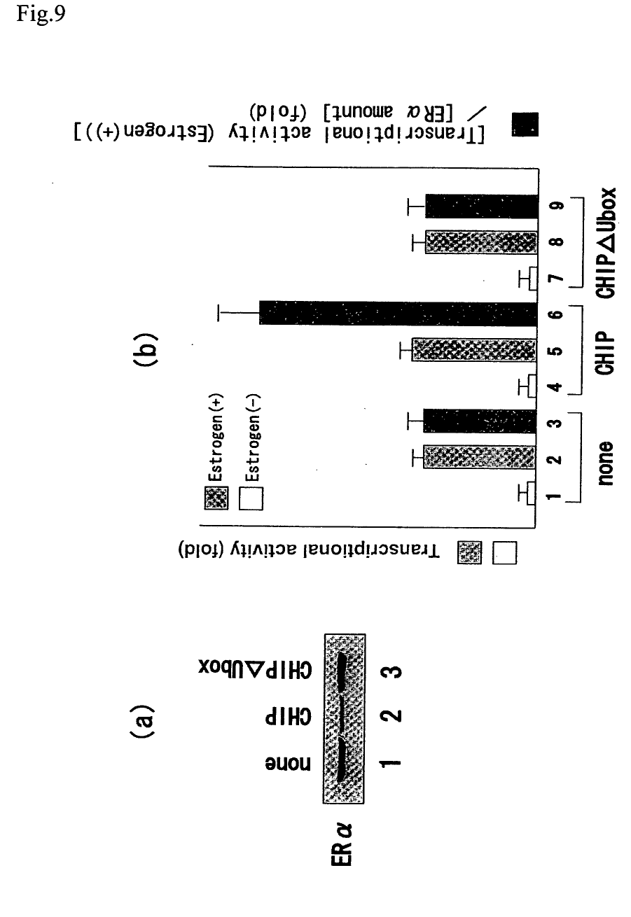

In the results, the amount of ERα fell markedly in the cells having introduced CHIP (see FIG. 9(a), lane 2). However, no diminution in the amount of ERα was observed in cells having introduced CHIPΔUbox (see FIG. 9(a), lane 3). Moreover, although the amount of ERα fell in the cells having introduced CHIP (see FIG. 9(a), lane 2), the transcriptional activation ability changed very little (see FIG. 9(b), lane 5). Consequently, transcriptional activation ability per amount of ERα rose (see FIG. 9(b), lane 6). There was no change in either transcriptional activation ability (FIG. 9(b), lane 8) or transcriptional activation ability per amount of ERα (FIG. 9(b), lane 9) in the cells having introduced CHIPΔUbox. The rise in transcriptional activation ability per amount of ERα may be attributable to selective degradation by CHIP of ERα the activity of which had been reduced by misfolding and changes over time. In FIGS. 9(a) and (b), “none” indicates results when neither CHIP nor CHIPΔUbox were introduced.

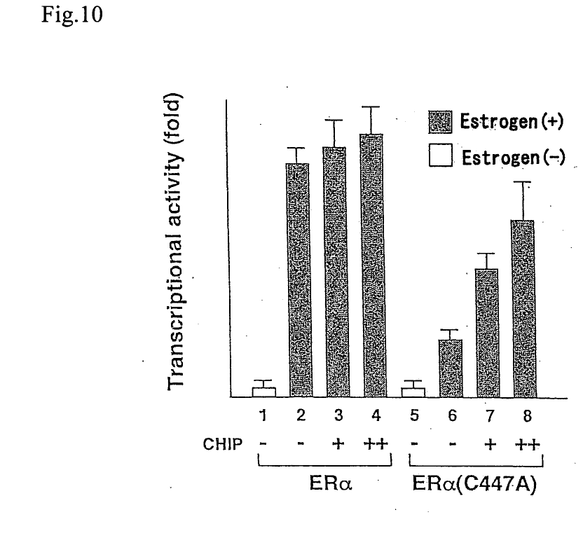

In cells having normal introduced ERα, there was a slight rise in transcriptional activation ability dependent on the amount of introduced CHIP (see FIG. 10, lanes 2 through 4), but when the temperature sensitive dominant negative mutant ERα(C447A) was introduced but CHIP was not introduced, the rise was dramatically less than when normal ERα was introduced (FIG. 10 lane 6). However, transcriptional activation ability rose significantly when CHIP was introduced (FIG. 10, lane 7 and 8). With ERα(C447A), which was less stable due to the mutation, the inactivated protein was strongly degraded while the protein retaining activity remained, eliminating the dominant negative effect and restoring transcriptional activation ability.

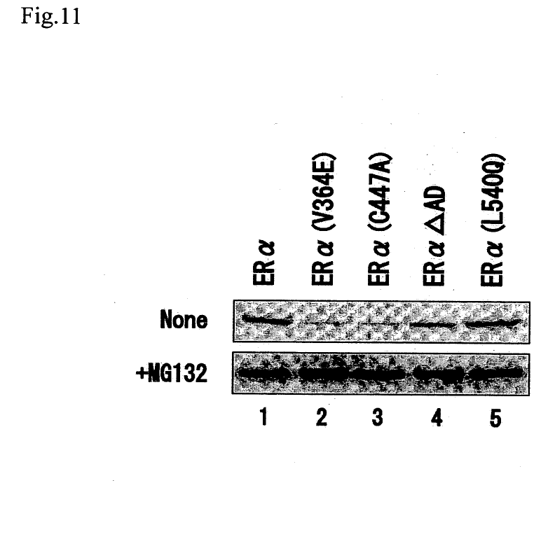

The amount of protein was significantly less for ERα(V364E) and ERα(C447A) than for normal ERα (see FIG. 11 lanes 2 and 3). The amount of protein was also slightly lower in the case of ERαΔAD (See FIG. 11 lane 4). However, when degradation of ERα was blocked by addition of the proteasome inhibitor MG132 to the cells, there was no difference between the various mutants and normal ERα in terms of amount of expression (see FIG. 11, lanes 1 through 5).

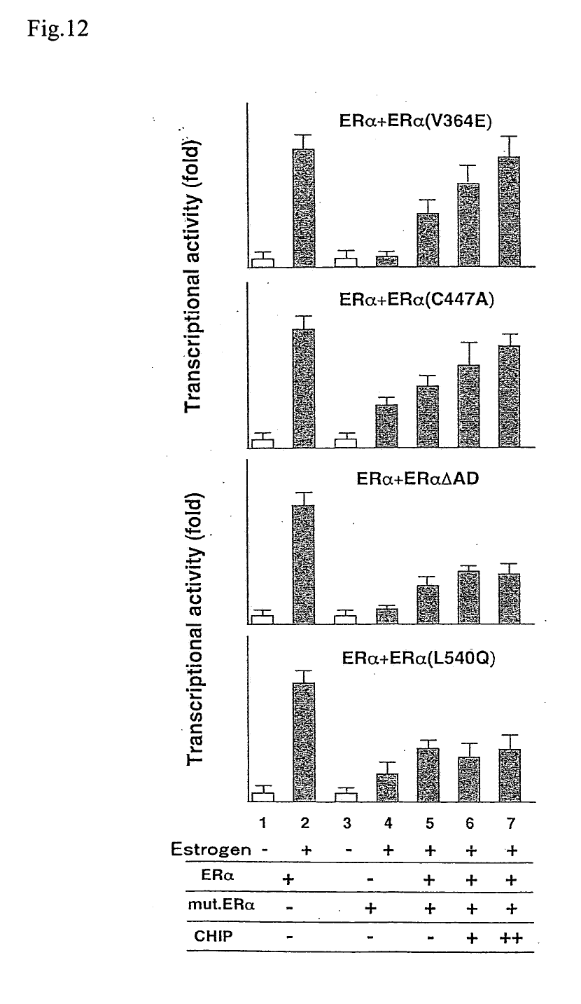

When normal ERα and mutant ERα (mut.ERα) were co-expressed without introduction of CHIP, transcriptional activation ability was reduced overall because the mutant ERα is dominant negative (see FIG. 12, lane 5). When CHIP was introduced, however, transcriptional activation ability in the case of ERα(V364E) and ERα(C447A) was restored dependent on the expressed amount of CHIP (see FIG. 12, lanes 6 and 7). This effect was not seen with ERαΔAD or ERα(L540Q) (see FIG. 12 lanes 6 and 7). In measurement of proteins by Western blotting, it was shown that in comparison with the normal form the two mutants are more selectively degraded. Dominant negative mutants depressed overall transcriptional activation ability when co-expressed with the normal form, but when CHIP was expressed in a mutant with marked selective degradation transcriptional activation ability was restored, while in the case of the two mutants in which degradation by CHIP was not marked there was little or no restoration of transcriptional activation ability.

From the results above it appears that estrogen-independent degradation of ERα by CHIP serves the function of maintaining the quality of intracellular ERα by removing ERα which has lost its original conformation and become inactive due to misfoldings during translation and changes over time.

1. A method for screening a substance which can suppress degradation of a nuclear receptor, comprising the steps of:
judging whether or not a test substance can suppress binding of a CHIP with a nuclear receptor via a chaperone protein; and
screening a test substance which can suppress the binding as a substance which can suppress degradation of a nuclear receptor.

2. A method for screening a substance which can suppress degradation of a nuclear receptor, comprising the steps of:
judging whether or not a test substance can suppress binding of a CHIP with a chaperone protein; and
screening a test substance which can suppress the binding as a substance which can suppress degradation of a nuclear receptor.

3. A method for screening a substance which can suppress degradation of a nuclear receptor, comprising the steps of:
judging whether or not a test substance can suppress binding of a chaperone protein with a nuclear receptor; and
screening a test substance which can suppress the binding as a substance which can suppress degradation of a nuclear receptor.

4. A method for screening a substance which can suppress degradation of a nuclear receptor, comprising the steps of:
judging whether or not a test substance can suppress ubiquitination of a nuclear receptor by CHIP; and
screening a test substance which can suppress the ubiquitination as a substance which can suppress degradation of a nuclear receptor.

5. A method for screening a substance which can suppress degradation of a nuclear receptor, comprising the steps of:
judging whether or not a test substance can suppress expression of a gene encoding a CHIP; and
screening a test substance which can suppress the expression as a substance which can suppress degradation of a nuclear receptor.

6. A method for screening a substance which can suppress degradation of a nuclear receptor, comprising the steps of:
judging whether or not a test substance can suppress expression of a gene encoding a chaperone protein; and
screening a test substance which can suppress the expression as a substance which can suppress degradation of a nuclear receptor.

7. A screening method according to any of claims 1 through 6, wherein the nuclear receptor is an estrogen receptor.

8. A screening method according to any of claims 1 through 6, wherein the substance which can suppress degradation of a nuclear receptor is a substance which can suppress nuclear receptor ligand-independent degradation of a nuclear receptor not bound to a nuclear receptor ligand.

9. A method for screening a substance which can promote degradation of a nuclear receptor, comprising the steps of:
judging whether or not a test substance can promote binding of a CHIP with a nuclear receptor via a chaperone protein; and
screening a test substance which can promote the binding as a substance which can promote degradation of a nuclear receptor.

10. A method for screening a substance which can promote degradation of a nuclear receptor, comprising the steps of:
judging whether or not a test substance can promote binding of a CHIP with a chaperone protein; and
screening a test substance which can promote the binding as a substance which can promote degradation of a nuclear receptor.

11. A method for screening a substance which can promote degradation of a nuclear receptor, comprising the steps of:
judging whether or not a test substance can promote binding of a chaperone protein with a nuclear receptor; and
screening a test substance which can promote the binding as a substance which can promote degradation of a nuclear receptor.

12. A method for screening a substance which can promote degradation of a nuclear receptor, comprising the steps of:
judging whether or not a test substance can promote ubiquitination of a nuclear receptor by CHIP; and
screening a test substance which can promote the ubiquitination as a substance which can promote degradation of a nuclear receptor.

13. A method for screening a substance which can promote degradation of a nuclear receptor, comprising the steps of:
judging whether or not a test substance can promote expression of a gene encoding a CHIP; and
screening a test substance which can promote the expression as a substance which can promote degradation of a nuclear receptor.

14. A method for screening a substance which can promote degradation of a nuclear receptor, comprising the steps of:
judging whether or not a test substance can promote expression of a gene encoding a chaperone protein; and
screening a test substance which can promote the expression as a substance which can promote degradation of a nuclear receptor.

15. A screening method according to any of claims 9 through 14, wherein the nuclear receptor is an estrogen receptor.

16. A screening method according to any of claims 9 through 14, wherein the substance which can promote degradation of a nuclear receptor is a substance which can promote nuclear receptor ligand-independent degradation of a nuclear receptor not bound to a nuclear receptor ligand.
